SEQUENCE LISTING (5 sequences)

SEQ ID NO: 1 - protein, 513 aa, Homo sapiens
MSSKPEPKDVHQLNGTGPSASPCSSDGPGREPLAGTSEFLGPDGAGVEVVIESRANAKGVREEDALLENGSQSNESDDVSTDRGPAPPSPLKETSFSIGLQVLFPFLLAGFGTVAAGMVLDIVQHWEVFQKVTEVFILVPALLGLKGNLEMTLASRLSTAANIGHMDTPKELWRMITGNMALIQVQATVVGFLASIAAVVFGWIPDGHFSIPHAFLLCASSVATAFIASLVLGMIMIGVIIGSRKIGINPDNVATPIAASLGDLITLALLSGISWGLYLELNHWRYIYPLVCAFFVALLPVWVVLARRSPATREVLYSGWEPVIIAMAISSVGGLILDKTVSDPNFAGMAVFTPVINGVGGNLVAVQASRISTFLHMNGMPGENSEQAPRRCPSPCTTFFSPDVNSRSARVLFLLVVPGHLVFLYTISCMQGGHTTLTLIFIIFYMTAALLQVLILLYIADWMVHWMWGRGLDPDNFSIPYLTALGDLLGTGLLALSFHVLWLIGDRDTDVGD

SEQ ID NO: 2 - RNA, 1542 nt, homo sapiens
auguccucuaagccagagccgaaggacguccaccaacugaacgggacuggcccuucugccucucccugcucuucagauggcccagggagagagcccuuggcugggaccucagaguuccuggggccugauggggcugggguagagguggugauugagucucgggccaacgccaaggggguucgggaggaggacgcccugcuggagaacgggagccagagcaacgaaagugacgacgucagcacagaccguggcccugcgccaccuuccccgcucaaggagaccuccuuuuccaucgggcugcaaguacuguuuccauuccuccuggcaggcuuugggaccguggcugcuggcaugguguuggacaucgugcagcacugggaagucuuccagaaggugacagaggucuucauccuagugccugcgcugcuggggcucaaagggaaccuggaaaugacccuggcaucaaggcuuuccacugcagccaacauuggacacauggacacacccaaggagcucuggcggaugaucacugggaacauggcccucauccaggugcaggccacgguggugggcuuccuggcguccaucgcagccgucgucuuuggcuggaucccugauggccacuucaguauuccgcacgccuuccugcucugugcuagcagcguggccacagccuucauugccucccugguacuggguaugaucaugauuggagucaucauuggcucucgcaagauugggaucaacccagacaacguggccacacccauugcugccagccugggcgaccucaucaccuuggcgcugcucucaggcaucagcuggggacucuaccuggaacugaaucacuggcgauacaucuacccacuggugugugcuuucuuuguggcccugcugccugucuggguggugcuggcccgacgaaguccagccacaagggagguguuguacucgggcugggagccuguuaucauugccauggccaucagcagugugggaggccucaucuuggacaagacugucucagaccccaacuuugcugggauggcugucuucacgccugugauuaaugguguugggggcaaucugguggcagugcaggccagccgcaucuccaccuuccugcacaugaauggaaugcccggagagaacucugagcaagcuccucgccgcugucccaguccuuguaccaccuucuucagcccugaugugaauucucgcucagcccggguccucuuccuccucguggucccaggacaccugguguuccucuacaccaucagcuguaugcagggcgggcacaccacccucacacucaucuucaucaucuucuauaugacagcugcacugcuccaggugcugauucuccuguacaucgcagacuggauggugcacuggauguggggccggggccuggacccggacaacuucuccaucccauacuugacugcucugggggaccugcuuggcacugggcuccuagcacucagcuuccauguucucuggcucauaggggaccgagacacggaugucggggacuag

SEQ ID NO: 3 - DNA, 1542 bp, homo sapiens
atgtcctctaagccagagccgaaggacgtccaccaactgaacgggactggcccttctgcctctccctgctcttcagatggcccagggagagagcccttggctgggacctcagagttcctggggcctgatggggctggggtagaggtggtgattgagtctcgggccaacgccaagggggttcgggaggaggacgccctgctggagaacgggagccagagcaacgaaagtgacgacgtcagcacagaccgtggccctgcgccaccttccccgctcaaggagacctccttttccatcgggctgcaagtactgtttccattcctcctggcaggctttgggaccgtggctgctggcatggtgttggacatcgtgcagcactgggaagtcttccagaaggtgacagaggtcttcatcctagtgcctgcgctgctggggctcaaagggaacctggaaatgaccctggcatcaaggctttccactgcagccaacattggacacatggacacacccaaggagctctggcggatgatcactgggaacatggccctcatccaggtgcaggccacggtggtgggcttcctggcgtccatcgcagccgtcgtctttggctggatccctgatggccacttcagtattccgcacgccttcctgctctgtgctagcagcgtggccacagccttcattgcctccctggtactgggtatgatcatgattggagtcatcattggctctcgcaagattgggatcaacccagacaacgtggccacacccattgctgccagcctgggcgacctcatcaccttggcgctgctctcaggcatcagctggggactctacctggaactgaatcactggcgatacatctacccactggtgtgtgctttctttgtggccctgctgcctgtctgggtggtgctggcccgacgaagtccagccacaagggaggtgttgtactcgggctgggagcctgttatcattgccatggccatcagcagtgtgggaggcctcatcttggacaagactgtctcagaccccaactttgctgggatggctgtcttcacgcctgtgattaatggtgttgggggcaatctggtggcagtgcaggccagccgcatctccaccttcctgcacatgaatggaatgcccggagagaactctgagcaagctcctcgccgctgtcccagtccttgtaccaccttcttcagccctgatgtgaattctcgctcagcccgggtcctcttcctcctcgtggtcccaggacacctggtgttcctctacaccatcagctgtatgcagggcgggcacaccaccctcacactcatcttcatcatcttctatatgacagctgcactgctccaggtgctgattctcctgtacatcgcagactggatggtgcactggatgtggggccggggcctggacccggacaacttctccatcccatacttgactgctctgggggacctgcttggcactgggctcctagcactcagcttccatgttctctggctcataggggaccgagacacggatgtcggggactag

SEQ ID NO: 4 - DNA, 1539 bp, Mus musculus
atgtcctctaagccagagccaaaggacatccaccagccgaatggtactggccctactccttctccctgttcttcggatggccctgggcgagagcccttggctgggacctcagagttcctggggcctgatggggtggaggtggtagtgattgagtcccgagctaacgccaagggaattcgggaggaggacgccctactggagaacgggagccagagtaacgagagtgacgatgtcagcacagaccggggccccgcgccaccctctccgctcaaggagacctccttttccattggactgcaagtgctgttccccttcctactggcaggctttgggactgtggcagctggcatggtgctggacattgtgcagcactgggaggtcttccagaaggtgacagaggtcttcatcctggtgcctgcactgctggggctcaaagggaacttggaaatgaccctggcgtcgaggctgtccactgcagccaacattggacaaatggacacacctaaggagctctggcggatgatcacggggaatatggcccttatccaggtacaagccacggtggtaggcttcctggcatccatcgcagccgtcgtctttggctggatccctgatggccactttagcatcccacacgccttcctgctatgcgccagcagtgtggctacagccttcattgcctccttggtcctgggtatgatcatgatcggagtcatcattgggtctcgaaagattgggatcaacccagacaacgtagccacgcccatcgccgctagcctgggcgacctcatcaccttggcactcctctcaggcatcagctggggactctacctggaactgaagcactggcgatacatctatcccctggtgtgtgccttctttgtggccctgctgcctgtctgggtggtgctggcccgaagaagcccggccacaagggaggtgttgtattcaggctgggagcctgtcatcattgctatggctatcagcagtgtgggaggtctcatcctggacaagaccgtctcggaccccaactttgcaggaatggctgtcttcacacctgtgattaatggtgtcgggggcaatctggtggctgtgcaggccagccgcatctccaccttcctccacatgaacggcatgccaggggagaactctgagccaactcctcgccgctgccccagtccatgcaccaccttcttcagccctgatgtgaattcacgctcggcccgtgtcctcttcctccttgtggtcccaggacacctagtgttcctctacaccatcagctgtatgcagggcgggcacaccaccctcaccctcatcttcatcatcttctacatgactgctgcactgctccaggtgctgattctcctgtacatcgcagactggatggtgcactggatgtggggccggggcctggaccccgacaacttctccatcccatacttgactgctttgggggacctgcttggcaccggactcctggcactcagcttccatgtgctctggctcattggggacagagacacagatgttggggactag

SEQ ID NO: 5 - protein, 512 aa, Mus musculus
MSSKPEPKDIHQPNGTGPTPSPCSSDGPGREPLAGTSEFLGPDGVEVVVIESRANAKGIREEDALLENGSQSNESDDVSTDRGPAPPSPLKETSFSIGLQVLFPFLLAGFGTVAAGMVLDIVQHWEVFQKVTEVFILVPALLGLKGNLEMTLASRLSTAANIGQMDTPKELWRMITGNMALIQVQATVVGFLASIAAVVFGWIPDGHFSIPHAFLLCASSVATAFIASLVLGMIMIGVIIGSRKIGINPDNVATPIAASLGDLITLALLSGISWGLYLELKHWRYIYPLVCAFFVALLPVWVVLARRSPATREVLYSGWEPVIIAMAISSVGGLILDKTVSDPNFAGMAVFTPVINGVGGNLVAVQASRISTFLHMNGMPGENSEPTPRRCPSPCTTFFSPDVNSRSARVLFLLVVPGHLVFLYTISCMQGGHTTLTLIFIIFYMTAALLQVLILLYIADWMVHWMWGRGLDPDNFSIPYLTALGDLLGTGLLALSFHVLWLIGDRDTDVGD

(19) United States
(12) Patent Application Publication
(10) Pub. No.: US 2013/0174289 A1
(43) Pub. Date: Jul. 4, 2013

(54) Na+/Mg2+ Exchanger

(71) Applicants: Monika Schweigel, Kuehlungs-Born (DE); Martin Kolisek, Berlin (DE)

(72) Inventors: Monika Schweigel, Kuehlungs-Born (DE); Martin Kolisek, Berlin (DE)

(73) Assignee: FBN - Leibnitz-Institut fur Nutztierbiologie, Dummerstorf (DE)

(21) Appl. No.: 13/823,383

(22) PCT Filed: Sep. 15, 2011
(86) PCT No.: PCT/EP11/65979

§ 371 Date: Mar. 14, 2013

(30) Foreign Application Priority Data
Sep. 18, 2010 (EP) 10177510.4

(51) Int. Cl. C07K 14/705; C12N 15/10

(52) U.S. Cl. CPC C07K 14/705; C12N 15/1079
USPC 800/14; 506/18; 435/354; 435/369; 435/366; 435/372.2

(57) ABSTRACT

The invention relates to methods to identify new SLC41A1 functions at the cell, tissue, organ and organism level. In part, it is related to methods useful in (a) identifying molecules that bind SLC41A1 polypeptides, which (b) modulate SLC41A1 related Na⁺/Mg²⁺ exchanger activity or its cellular or tissue specific expression. Thus, the invention comprises SLC41A1 mutation libraries, SLC41A1 specific antibodies, their generation and finding as well as an inducible conditional SLC41A1 knock out mice model and inducible conditional knock out SLC41A1 cell lines.

The magnesium ion Mg²⁺ is a vital element essentially involved in numerous physiological processes at the cell, organ and organism level [Kolisek et al., 2007]. Therefore, the biologically active free intracellular Mg²⁺ concentration ([Mg²⁺]ᵢ) has to be adjusted to its optimal level of 0.5 to 1.2 mM. In this process transmembrane extrusion of Mg²⁺ has been shown to be critically involved (Schweigel et al., 2006). So far, there is no evidence for the existence of an outwardly oriented Mg-ATPase, however, by functional studies two main efflux systems, namely an anion-dependent mechanism [Ödblom and Handy, 1999] and a putative Na⁺/Mg²⁺ exchanger [Schweigel et al., 2006, 2009], have been proposed. After its first description in chicken and turkey red blood cells (Günther et al., 1984), Na⁺-dependent efflux of intracellular Mg²⁺ has been shown to operate in the majority of examined cells including ventricular myocytes, vascular smooth muscle cells, hepatocytes, thymocytes, neurons, renal and gastrointestinal epithelial cells (Kolisek et al., 2007; Schweigel et al., 2006). The Na⁺/Mg²⁺ exchanger is also a candidate mechanism for the extrusion of Mg²⁺ across the basolateral membrane of the Mg²⁺ absorbing and reabsorbing intestinal and renal epithelia (Quamme, 1989) making it important for control of body Mg²⁺ homeostasis (balance). The near-ubiquity of the Na⁺/Mg²⁺ exchanger attests to its functional importance, as is substantiated by its participation in the pathogenesis of a growing collection of human diseases (Kimura, 2007). Disturbance or dysregulation of the antiporter seem to be involved in primary hypertension, ischemic heart disease, eclampsia, diabetes and cystic fibrosis to name a few (Kisters et al., 1999; Vormann et al., 1992; Kimura, 2007; Kumeda and Inaba, 2007).

All approaches used so far, in order to identify the encoding gene(s) and/or to solve the protein structure of the Na⁺/Mg²⁺ exchanger, e.g. establishment of a high (>100 mM)-Mg²⁺ resistant cell line (Watanabe et al., 2005), failed. Only the protein structure of the Na⁺/Mg²⁺ exchanger will allow for a detailed investigation of the modalities of its operation and regulation as well as for the development of specific inhibitors or agonists.

Within the last years several candidate genes have been characterized encoding for proteins involved in the regulation of eukaryotic cells Mg²⁺ homeostasis (summarized in [Sponder et al., 2010]). Only few of them have been clearly shown to be directly and specifically involved in Mg²⁺ transport processes. The best characterized are the TRPM6 and TRPM7 channels of the Melastatin-related Transient Receptor Potential family considered as being the major Mg²⁺ influx pathways of mammalian cells and the mitochondrial channel MRS2 (Mitochondrial RNA Splicing member 2) that mediates Mg²⁺ influx into the mitochondrial matrix [Schmitz, 2003; Kolisek, 2003, Schlingmann et al., 2002]. Other putative Mg²⁺ transporters, found by a differential gene expression approach (Goytain and Quamme, 2005a-d, Goytain et al., 2007), are MagT1 (Magnesium Transporter 1), ACDP 2 (Ancient Conserved Domain Protein; subtype 2), the protein NIPA1 (Nonimprinted in Prader-Willi/Angelman), SLC41A1 and SLC41A2 (Solute Carrier family 41; subfamily A; members 1 and 2).

When over-expressed in Xenopus laevis oocytes, a genetically heterologous system, the group of Quamme [Goytain and Quamme, 2005a-d; Goytain et al., 2007] characterized mouse (m)SLC41A1, mSLC41A2, mACDP 2 and mMagT1 all as being Mg²⁺ channels mediating influx of the ion. However, with the exception of MagT1 the transport was not very specific to Mg²⁺ and, when over-expressed in human-derived cells hSLC41A2 [ Sahni et al., 2007], hMagT1 [ Zhou and Clapham, 2009] and hSLC41A1 (Kolisek et al., 2008) all failed to induce Mg²⁺ evoked currents. Moreover, by using tet-induced over-expression of hSLC41A1 in HEK293 cells strong evidence was found for human SLC41A1 as being a Mg²⁺ carrier localized to the cytoplasmatic membrane and mediating efflux of the ion [Kolisek et al., 2008]. SLC41A1 has been shown to have partial sequence homology [Wabakken et al., 2003] to a specific region in the transmembrane domain of the bacterial Mg²⁺ transport system MgtE described by Smith et al. (1995). It is interesting to note, that the MgtE proteins have been discovered by complementation assays and that they show no homology to other groups of bacterial Mg²⁺ transporters operating as pumps (MgtA, MgtB) or channels (CorA). Human [Wabbaken et al., 2003] and mice [Goytain and Quamme, 2005c] SLC41A1 transcripts have been found to be highly abundant in heart, brain, kidney, liver, muscle, testis, thyroid gland, lymphocytes and to a lesser extent in most other tissues including colon and small intestine. Neither the functions of MgtE nor of SLC41A1 have been thoroughly characterized.

Thus, the object of the present invention was the identification of the encoding gene(s) and the salvation of the protein structure of the Na⁺/Mg²⁺ exchanger.

The object was solved by the finding that the protein SLC41A1 is the cellular Na⁺/Mg²⁺ exchanger. It could be proven that the protein SLC41A1 mediates Mg²⁺ extrusion and its mode of operation could be clarified. In the following, the naming SEQ ID NO 1 is used for the protein SLC41A1 (GenBank Accession No. NP 776253) and the naming SEQ ID NO 2 is used for the underlying mRNA sequence of SLC41A1 (GenBank Accession No. NM_{—}173854). The cDNA sequence is represented in SEQ ID NO 3. SEQ ID NO 4 represents the SLC41A1 from mouse (Mus musculus, complete cds, GenBank Accession No. BC116280.1, CDS: 802>2340) and SEQ ID NO 5 is the related protein sequence (Mus musculus, NCBI Reference Sequence: NP_{—}776290.1).

The invention relates to methods to identify new SLC41A1 functions at the cell, tissue, organ and organism level. In part, it is related to methods useful in (a) identifying molecules that bind SLC41A1 polypeptides, which (b) modulate SLC41A1 related Na⁺/Mg²⁺ exchanger activity or its cellular or tissue specific expression.

Thus, the invention comprises SLC41A1 mutation libraries, SLC41A1 specific antibodies and their generation as well as an inducible conditional knock out mice model and a conditional inducible knock out cell line.

The SLC41A1 mutation libraries comprise at least one of the following mutation clusters i, ii, iii, iv of the amino acid sequence of SEQ ID NO 1 (indicated in brackets are the triplet changes of the cDNA of SEQ ID NO 3):
i) One mutation library comprises site-directed mutants of SEQ ID NO 1, wherein a single amino acid (aa) is substituted in position: 21S, 25S, 73S, 76S, 80S, 89S, 97S, 158S, 309S, 394S, 408S, 81T, 168T, 509T and 278Y, respectively.
   In other words, a single aa serine (S), threonine (T) or tyrosine (Y) is replaced at one of the following positions of SEQ ID NO 1 by another aa:
   21 S>N (TCT>AAT), 25 S>Q (TCA>CAA), 73 S>N (AGC>AAC), 76 S>N (AGT>AAT), 80 S>N (AGC>AAC), 89 S>N (TCC>AAC), 97 S>N (TCC>AAC), 158 S>N (TCC>AAC), 309 S>N (AGT>AAT), 394 S>N (AGT>AAT) and 408 S>Q (TCA>CAA), 81 T>Q (ACA>CAA), 168 T>Q (ACA>CAA), 509 T>Q (ACG>CAG) and 278 Y>N (TAC>AAC), respectively. Comprised are also combinations of these mutations, i.e. at least one mutation of SEQ ID NO 1, wherein a plurality (2-15) of these substitutions is present. Preferably, 2-5 aa's are replaced, most preferably 2 or 3. In other words, the mutation library of cluster i) consists of 15 peptides with single aa mutation and at least one mutation of SEQ ID NO 1, wherein a plurality (2-15) of these substitutions is present.
ii) Another mutation library comprises mutations of the transmembrane domains (TM) and N-, C-termini mutants of SEQ ID NO 1:
   Based on SEQ ID NO 1, respective deletions (Δ) of the amino acid sequences were present in:
   a) N-terminus and C-terminus flanking region: Δaa 2-30, Δaa 31-60, Δaa 61-90 and Δaa 2-60, Δaa 2-90, Δaa 504-513, respectively (6 mutated proteins)
   and
   b) membrane spanning domains of SEQ ID NO 1 of highly probable TM10 model (TMP): Δaa 96-205 (TM1+TM2); Δaa 225-279 (TM3+TM4); Δaa 286-337(TM5+TM6); Δaa 348-427 (TM7+TM8) and Δaa 439-503 (TM9+TM10), respectively (5 mutated proteins)
   and
   c) membrane spanning domains of less-probable TM11 model (SOSUI): Δaa 99-150 (TM1+TM2); Δaa 180-241 (TM3+TM4); Δaa 256-306 (TM5+TM6); Δaa 317-368 (TM7+TM8); Δaa 408-459 (TM9+TM10) and Δaa 437-503 (TM10+TM11), respectively (6 mutated proteins).
   Overall, cluster ii) comprises 17 mutated proteins.
iii) Another mutation library comprises leucine zipper (LZ) mutants of SEQ ID NO 1:
   These SLC41A1 mutants comprise a deletion (Δ) respective to SEQ ID NO 1 of
   a) Δaa 482-503 and
   b) aa substitutions in LZ domain in positions 482 L>V (TTG>GTG), 489 L>V (CTT>GTT), 494 L>V (CTA>GTA), 496 L>V (CTC>GTC), 501 L>V (CTC>GTC) and 503 L>V (CTC>GTC), respectively (7 mutated proteins).
iv) Another mutation library comprises external loops (EL) mutants of SEQ ID NO 1:
   These SLC41A1 mutants comprise deletions (A) respective to SEQ ID NO 1 of the amino acid sequences in EL: Δaa 128-179, Δaa 247-254, Δaa 310-315, Δaa 369-405 and Δaa 468-478 respectively (5 mutated proteins).

The mutations described in cluster i) are believed to be useful for the identification of SLC41A1 variants showing a modified phosphorylation pattern and therefore, an impaired or modified function of the carrier.

Mutations in the membrane spanning domains (cluster ii) and EL mutations (cluster iv) are believed to be useful for the identification of the minimal sequential configuration necessary to maintain the Mg²⁺/Na⁺ exchange (efflux) function of SLC41A1 in vivo and of the Mg²⁺ binding motif(s)/the Mg²⁺ binding site. Thus, they will be helpful to generate more active SLC41A1 variants or SLC41A1 variants with impaired function of the SLC41A1 carrier.

The LZ domain is known to be critical in dimerization and thus, for the complex forming ability of SLC41A1 which is essential for its normal function as a carrier (Kolisek et al., 2008). Mutations in the LZ domain (cluster iii) are believed to be helpful in the generation of SLC41A1 variants with changed transport and regulatory characteristics and/or mode of action.

Each of these sequences is part of the present invention. Preferably, they are combined in a mutation library, which comprises at least all sequences of one of the mutation clusters i) to iv). The most preferred embodiment is a mutation library, which combines all four mutation clusters i) to iv).

The expected outcome of mutation clusters i, ii and iv is also a definitive confirmation of the SLC41A1 orientation in cytoplasmatic membrane of the cell. As shown by Kolisek et al. (2008), a predicted model which comprises 10 TMs and both N- and C-terminus oriented intracellularly is most likely.

Respective mutated SLC41A1 inserts (from cluster i, ii, iii and/or iv) are cloned into cloning vector pUC19, the selected clones are controlled for the proper insert orientation and sequenced for verification purposes. The constructed library is subcloned into respective mammalian expression vector(s) and used for functional screening in an inducible conditional SLC41A1 knockout cell line or into cells of a transgenic conditional knockout animal, which both will be described in more detail below.

Furthermore, the present invention is related in one aspect to the use of a protein consisting of an amino acid sequence SEQ ID NO 1 or a protein fragment thereof, in which one or more amino acids are substituted, deleted or added and which has an identity on the amino acid level with SEQ ID NO 1 of at least 90%, preferably at least 95%, more preferably 98%, for the generation and identification of specific antibodies against the cellular Na⁺/Mg²⁺ exchanger.

Below, the term “identity of at least 90%” always means that this comprises preferably an identity of at least 95%, more preferably an identity of 98%, regardless whether proteins or DNAs are concerned.

The related method of identifying antibodies against the cellular Na⁺/Mg²⁺ exchanger comprises the steps of:
contacting a protein having SEQ ID NO 1 or a protein fragment thereof, in which one or more amino acids are substituted, deleted or added and which has an identity on the amino acid level with SEQ ID NO 1 of at least 90%
with a primary antibody under conditions that allow complex formation of the primary antibody with the protein or the protein fragment and
detecting the complex.

The complex is preferably detected by a secondary antibody. In a preferred embodiment, the primary and/or the secondary antibody comprise(s) a label, which is selected from the group of radioactive label, fluorescent label, enzymatic label, or biotin label.

Another aspect of the present invention is related to a kit for identifying antibodies against the Na⁺/Mg²⁺ exchanger, wherein the kit comprises a protein consisting of an amino acid sequence of SEQ ID NO 1 or a protein fragment thereof, in which one or more amino acids are substituted, deleted or added and which has an identity on the amino acid level with SEQ ID NO 1 of at least 90%.

Additionally, the present invention is related to the use of a protein consisting of an amino acid sequence of SEQ ID NO 1 or a protein fragment thereof, in which one or more amino acids are substituted, deleted or added and which has an identity on the amino acid level with SEQ ID NO 1 of at least 90% in a method which uses the protein in order to generate and produce antibodies, preferably monoclonal antibodies, against the Na⁺/Mg²⁺ exchanger.

Several antibodies were provided which selectively bind to the Na⁺/Mg²⁺ exchanger. A special embodiment of the invention is related to seven antibodies, wherein each specifically recognises one of the following epitopes

ESRANAKGVREEDALLENGS,
ESDDVSTDRGPAPPSPLKE,
LKGNLEMTLASRLSTAAN,
STAANIGHMDTPKEL,
SSVGGLILDKTVSDPN,
PGENSEQAPRRCPSPCTT
or
ADWMVHWMWGRGLDPDN of SEQ ID NO 1.

These antibodies can be used for the detection of the presence, absence and or amount and for the interaction with the cellular Na⁺/Mg²⁺ exchanger, which has a protein having the amino acid sequence of SEQ ID NO 1 or a protein fragment thereof, in which one or more amino acids are substituted, deleted or added and which has an identity on the amino acid level with SEQ ID NO 1 of at least 90%. The antibodies may be polyclonal or monoclonal but monoclonal antibodies are preferred.

Another aspect of the present invention is related to the use of a protein consisting of an amino acid sequence of SEQ ID NO 1 or a protein fragment thereof, in which one or more amino acids are substituted, deleted or added and which has an identity on the amino acid level with SEQ ID NO 1 of at least 90% for the identification of specific protein binding partners that can form stable or transient protein complexes with the cellular Na⁺/Mg²⁺ exchanger.

The related method of identifying specific protein complex partners for the Na⁺/Mg²⁺ exchanger, comprises the steps of:
contacting a cell, which contains a protein of amino acid sequence SEQ ID NO 1 or a fragment thereof, in which one or more amino acids are substituted, deleted or added and which has an identity on the amino acid level with SEQ ID NO 1 of at least 90%
with a candidate substance under conditions that allow for an interaction of the candidate substance with the protein or the fragment,
determining the free or total intracellular Mg²⁺ concentration or the extracellular Mg²⁺ concentration, and
comparing the Mg²⁺ concentration as determined with the free or total intracellular Mg²⁺ concentration or the extracellular Mg²⁺ concentration in the absence of the candidate substance.

Another aspect of the invention is related to a kit for identifying specific protein binding partners forming stable or transient protein complexes with the Na⁺/Mg²⁺ exchanger, wherein the kit comprises a protein having an amino acid sequence of SEQ ID NO 1 or a protein fragment thereof, in which one or more amino acids are substituted, deleted or added and which has an identity on the amino acid level with SEQ ID NO 1 of at least 90%.

Especially the substances listed belowprove suitable as protein complex partner for the Na⁺/Mg²⁺ exchanger, which is identical to a protein having an amino acid sequence of SEQ ID NO 1 or a protein fragment thereof, in which one or more amino acids are substituted, deleted or added and which has an identity on the amino acid level with SEQ ID NO 1 of at least 90%:

3-beta-hydroxysteroid-delta(8),delta(7)-isomerase (OS=homo sapiens, UniProt ID Q15125); 3-beta-hydroxysteroid-delta(8),Delta(7)-isomerase OS=Rattus norvegicus, UniProt ID Q9JJ46); ATP synthase subunit a (OS=Cricetulus griseus, UniProt ID P14413); ATP synthase subunit a (OS=homo sapiens, UniProt ID P00846); ATP synthase subunit a (OS=Pan troglodytes, UniProt ID Q9T9W0); ATP synthase subunit a (OS=Tachyglossus aculeatus aculeatus, UniProt ID Q8W9G8); B-cell receptor-associated protein 31 (OS=Homo sapiens, UniProt ID P51572); Immediate early response 3-interacting protein 1 (OS=Homo sapiens, UniProt ID Q9Y5U9); Keratinocyte-associated protein 2 (OS=Homo sapiens, UniProt ID Q8N6L1); Kunitz-type protease inhibitor 2 (OS=Homo sapiens, UniProt ID 043291); Peptidyl-prolyl cis-trans isomerase B (OS=Homo sapiens, UniProt ID P23284); Probable ergosterol biosynthetic protein 28 (OS=Pongo abelii, UniProt ID Q5R589); Protein disulfide-isomerase A6 (OS=Homo sapiens, UniProt ID Q15084); Protein YIPF6 (OS=Homo sapiens, UniProt ID Q96EC8); Putative uncharacterized protein CXorf62 (OS=Homo sapiens, UniProt ID Q8N2A0); Signal peptidase complex subunit 1 (OS=Pongo abelii, UniProt ID Q5RF96); Stress-associated endoplasmic reticulum protein 1 (OS=Rattus norvegicus, UniProt ID Q9R2C1); Translocon-associated protein subunit gamma (OS=Homo sapiens, UniProt ID Q9UNL2); Transmembrane protein 147 (OS=Homo sapiens, UniProt ID Q9BVK8); Transmembrane protein 14A (OS=Homo sapiens, UniProt ID Q9Y6G1); Uncharacterized protein C10 or 135 (OS=Homo sapiens, UniProt ID Q96D05); Uncharacterized protein C4orf3 (OS=Homo sapiens, UniProt ID Q8WVX3); UPF0414 transmembrane protein C20orf30 (OS=Homo sapiens, UniProt ID Q96A57); UPF0480 protein C15orf24 homolog (OS=Macaca fascicularis, UniProt ID Q4R5V2); and UPF0480 protein C15orf24 (OS=Homo sapiens, UniProt ID Q9NPA0).

Preferred molecules out of these are 3-beta-hydroxysteroid-delta(8),delta(7)-isomerase (OS=Homo sapiens, UniProt ID Q15125); B-cell receptor-associated protein 31 (OS=Homo sapiens, UniProt ID P51572); Peptidyl-prolyl cis-trans isomerase (OS=Homo sapiens, UniProt ID P23284) and UPF0480 protein C15orf24 (OS=Macaca fascicularis, UniProt ID Q4R5V2).

These SLC41A1 complex-forming partners were screened out by the split-ubiquitin method, which is well known in the art and are listed in detail in Table 1. The ubiquitin-based split protein sensor (USPS) is the method that makes it possible to detect and monitor protein-protein interactions as a function of time, at the natural sites of these interactions in a living cell. Details on the method can be found in Johnsson and Varshaysky, 1994.

The more often the screened molecules occur in the list given in Table 1, the higher is their ability of being a binding partner for SLC41A1, i.e. the more clones revealed interaction with SLC41A1. In order to shorten the table, multiple entries were deleted and only the frequency of occurrence (Occurrence) is indicated as a number. The singletons listed in Table 2 are the weakest partners. They are organized from the strongest to the weakest.

TABLE 1

| protein name (description) | uniprot ID | occurrence |
|---|---|---|
| 3-beta-hydroxysteroid-Delta(8), Delta(7)-isomerase OS = Homo | Q15125 | 24 |
| sapiens GN = EBP PE = 1 SV = 3 | | |
| 3-beta-hydroxysteroid-Delta(8), Delta(7)-isomerase OS = Rattus | Q9JJ46 | 1 |
| norvegicus GN = Ebp PE = 2 SV = 3 | | |
| ATP synthase subunit a OS = Cricetulus griseus GN = MT-ATP6 | P14413 | 1 |
| PE = 3 SV = 1 | | |
| ATP synthase subunit a OS = Homo sapiens GN = MT-ATP6 | P00846 | 1 |
| PE = 1 SV = 1 | | |
| ATP synthase subunit a OS = Pan troglodytes GN = MT-ATP6 | Q9T9W0 | 1 |
| PE = 3 SV = 1 | | |
| ATP synthase subunit a OS = Tachyglossus aculeatus aculeatus | Q8W9G8 | 1 |
| GN = MT-ATP6 PE = 3 SV = 1 | | |
| B-cell receptor-associated protein 31 OS = Homo sapiens | P51572 | 10 |
| GN = BCAP31 PE = 1 SV = 3 | | |
| Immediate early response 3-interacting protein 1 OS = Homo | Q9Y5U9 | 3 |
| sapiens GN = IER3IP1 PE = 1 SV = 1 | | |
| Keratinocyte-associated protein 2 OS = Homo sapiens | Q8N6L1 | 2 |
| GN = KRTCAP2 PE = 1 SV = 1 | | |
| Kunitz-type protease inhibitor 2 OS = Homo sapiens | O43291 | 2 |
| GN = SPINT2 PE = 1 SV = 2 | | |
| Peptidyl-prolyl cis-trans isomerase B OS = Homo sapiens | P23284 | 5 |
| GN = PPIB PE = 1 SV = 2 | | |
| Probable ergosterol biosynthetic protein 28 OS = Pongo abelii | Q5R589 | 2 |
| PE = 2 SV = 1 | | |
| Protein disulfide-isomerase A6 OS = Homo sapiens GN = PDIA6 | Q15084 | 11 |
| PE = 1 SV = 1 | | |
| Protein YIPF6 OS = Homo sapiens GN = YIPF6 PE = 2 SV= 2 | Q96EC8 | 2 |
| Putative uncharacterized protein Cxorf62 OS = Homo sapiens | Q8N2A0 | 2 |
| GN = Cxorf62 PE = 2 SV = 1 | | |
| Signal peptidase complex subunit 1 OS = Pongo abelii | Q5RF96 | 2 |
| GN = SPCS1 PE = 3 SV = 1 | | |
| Stress-associated endoplasmic reticulum protein 1 OS = Rattus | Q9R2C1 | 4 |
| norvegicus GN = Serp1 PE = 1 SV = 2 | | |
| Translocon-associated protein subunit gamma OS = Homo | Q9UNL2 | 4 |
| sapiens GN = SSR3 PE = 1 SV = 1 | | |
| Transmembrane protein 147 OS = Homo sapiens | Q9BVK8 | 2 |
| GN = TMEM147 PE = 1 SV = 1 | | |
| Transmembrane protein 14A OS = Homo sapiens | Q9Y6G1 | 2 |
| GN = TMEM14A PE = 2 SV = 1 | | |
| Uncharacterized protein C10orf35 OS = Homo sapiens | Q96D05 | 2 |
| GN = C10orf35 PE = 1 SV = 1 | | |
| Uncharacterized protein C4orf3 OS = Homo sapiens GN = C4orf3 | Q8WVX3 | 2 |
| PE = 2 SV = 1 | | |
| UPF0414 transmembrane protein C20orf30 OS = Homo sapiens | Q96A57 | 3 |
| GN = C20orf30 PE = 1 SV = 1 | | |
| UPF0480 protein C15orf24 homolog OS = Macaca fascicularis | Q4R5V2 | 1 |
| GN = QtsA-20627 PE = 2 SV = 1 | | |
| UPF0480 protein C15orf24 OS = Homo sapiens GN = C15orf24 | Q9NPA0 | 4 |
| PE = 1 SV = 1 | | |

Further Singletons are listed below in table 2.

TABLE 2

| | UniProt |
|---|---|
| Protein name (description) | ID |
| ADP-ribosylation factor-like protein 6-interacting protein | Q66PJ3 |
| 4 OS = Homo sapiens GN = ARL6IP4 PE = 1 SV = 1 | |
| Elongation of very long chain fatty acids protein 4 | Q9GZR5 |
| OS = Homo sapiens GN = ELOVL4 PE = 2 SV = 1 | |
| Proteolipid protein 2 OS = Homo sapiens GN = PLP2 | Q04941 |
| PE = 1 SV = 1 | |
| Putative P2Y purinoceptor 10 OS = Homo sapiens | O00398 |
| GN = P2RY10 PE = 2 SV = 1 | |
| Transmembrane protein 106C OS = Homo sapiens | Q9BVX2 |
| GN = TMEM106C PE = 2 SV = 1 | |
| Solute carrier family 35 member B1 OS = Homo sapiens | P78383 |
| GN = SLC35B1 PE = 2 SV = 1 | |
| Thioredoxin-related transmembrane protein 1 OS = Homo | Q9H3N1 |
| sapiens GN = TMX1 PE = 1 SV = 1 | |
| Transmembrane emp24 domain-containing protein 10 | P49755 |
| OS = Homo sapiens GN = TMED10 PE = 1 SV = 2 | |
| Putative uncharacterized protein C3orf66 OS = Homo | Q6ZWE1 |
| sapiens GN = C3orf66 PE = 5 SV = 1 | |
| Probable phospholipid-transporting ATPase ID OS = Homo | P98198 |
| sapiens GN = ATP8B2 PE = 1 SV = 2 | |
| Signal recognition particle 9 kDa protein OS = Homo sapiens | P49458 |
| GN = SRP9 PE = 1 SV = 2 | |
| Zinc transporter ZIP13 OS = Rattus norvegicus | Q2M1K6 |
| GN = SIc39a13 PE = 2 SV = 1 | |
| Phosphatidate phosphatase PPAPDC1B OS = Mus musculus | Q3UMZ3 |
| GN = Ppapdc1b PE = 2 SV = 1 | |
| WW domain-binding protein 11 OS = Rattus norvegicus | Q5PQQ2 |
| GN = Wbp11 PE = 2 SV = 1 | |
| Emerin OS = Homo sapiens GN = EMD PE = 1 SV = 1 | P50402 |
| Transmembrane 6 superfamily member 1 OS = Homo sapiens | Q9BZW5 |
| GN = TM06F1 PE = 2 SV = 2 | |
| C-4 methylsterol oxidase OS = Pongo abelii GN = SC4MOL | Q5R574 |
| PE = 2 SV = 1 | |
| Single-stranded DNA-binding protein 2 OS = Homo sapiens | P81877 |
| GN = SSBP2 PE = 1 SV = 2 | |
| Putative uncharacterized protein C14orf165 OS = Homo | Q86U02 |
| sapiens GN = C14orf165 PE = 4 SV = 1 | |
| Zinc finger protein 714 OS = Homo sapiens | Q96N38 |
| GN = ZNF714 PE = 2 SV = 1 | |
| Calcium release-activated calcium channel protein 1 | Q96D31 |
| OS = Homo sapiens GN = ORAI1 PE = 1 SV = 2 | |
| Transmembrane BAX inhibitor motif-containing protein 4 | Q9HC24 |
| OS = Homo sapiens GN = TMBIM4 PE = 1 SV = 2 | |
| NADH-ubiquinone oxidoreductase chain 3 OS = Homo | P03897 |
| sapiens GN = MT-ND3 PE = 1 SV = 1 | |
| ADP-ribosylation factor-like protein 6-interacting protein 6 | Q8N065 |
| OS = Homo sapiens GN = ARL6IP6 PE = 1 SV = 1 | |
| Ribonuclease kappa OS = Mus musculus GN = Rnasek | Q8K3C0 |
| PE = 2 SV = 1 | |
| Wiskott-Aldrich syndrome protein homolog OS = Mus | P70315 |
| musculus GN = Was PE = 1 SV = 1 | |
| Cytochrome c oxidase subunit 2 OS = Homo sapiens | P00403 |
| GN = MT-CO2 PE = 1 SV = 1 | |
| Melanoma-associated antigen D1 OS = Mus musculus | Q9QYH6 |
| GN = Maged1 PE = 1 SV= 1 | |
| Probable low affinity copper uptake protein 2 OS = Homo | O15432 |
| sapiens GN = SLC31A2 PE = 2 SV = 1 | |
| Protein comichon homolog 4 OS = Pongo abelii | Q5R9M4 |
| GN = CNIH4 PE = 2 SV = 1 | |
| Mitochondrial carrier homolog 2 OS = Pongo abelii | Q5R5M0 |
| GN = MTCH2 PE = 2 SV = 2 | |
| Squalene synthetase OS = Pongo abelii GN = FDFT1 PE = 2 | Q5R6U3 |
| SV = 1 | |
| RING finger protein 114 OS = Pan troglodytes GN = RNF114 | Q6J212 |
| PE = 2 SV = 1 | |
| Interferon alpha-inducible protein 6 OS = Homo sapiens | P09912 |
| GN = IFI6 PE = 2 SV = 2 | |
| Protein tyrosine phosphatase receptor type C-associated | Q14761 |
| protein OS = Homo sapiens GN = PTPRCAP PE = 1 SV = 1 | |
| Protein FAM192A OS = Homo sapiens GN = FAM192A | Q9GZU8 |
| PE = 1 SV = 1 | |
| Intercellular adhesion molecule 2 OS = Homo sapiens | P13598 |
| GN = ICAM2 PE = 1 SV = 2 | |
| Leptin receptor gene-related protein OS = Homo sapiens | O15243 |
| GN = LEPROT PE = 1 SV = 1 | |

Thus, another aspect of the invention is related to the use of said molecules as protein complex partners for the Na⁺/Mg²⁺ exchanger thereby regulating or modifying its function. Additionally, these specific protein binding partners are intended for the use in therapeutical applications, especially for the treatment of the diseases mentioned below (Table 4).

In the above tables 1 and 2 underlined names indicate highly connected proteins, found in the majority of DUALmembrane SU-YTH screens. Highly connected proteins include chaperones, stress-induced endoplasmatic reticulum proteins, components of the translocon machinery and proteins involved in membrane protein sorting and transport.

The most prominent candidate binding partner is 3-beta-hydroxysteroid-Delta(8),Delta(7)-isomerase, an enzyme which catalyzes the conversion of Delta(8)-sterols to their corresponding Delta(7)-isomers. In the screening 24 clones were identified where 3-beta-hydroxysteroid-Delta(8),Delta(7)-isomerase interacted with SLC41A1. Defects in 3-beta-hydroxysteroid-Delta(8),Delta(7)-isomerase are the cause of chondrodysplasia punctata also known as Conradi-Hunermann-Happle syndrome (Derry et al, 1999). As for the other candidate binding partners identified, the nature of interaction between SLC41A1 and 3-beta-hydroxysteroid-Delta(8),Delta(7)-isomerase is unknown. Its identification will help to understand the link between disturbed cholesterol metabolism and magnesium insufficiency in mammals.

Ten clones revealed interaction between SLC41 and B-cell receptor associated-protein 31. It is expected to play a role in anterograde transport of membrane proteins from the endoplasmic reticulum to the Golgi. It may be also involved in CASP8-mediated apoptosis.

The third strongest SLC41A1-binding candidates were Peptidyl-prolyl cis-trans isomerase and UPF0480 protein C15orf24.

The rest of the identified SLC41A1 binding partners, found in more than only one clone, is merely represented by poorly characterized or uncharacterized transmembrane proteins, proteins involved in mediating the cellular response to mitogenic signals (immediate early-response 3-interacting protein; SABiosciences) and endoplasmatic reticulum-located probable ergosterol biosynthetic protein 28, transmembrane protein 147 and APH-1-similar core component of nicalin-nomo complexes. One interesting candidate is the Kunitz type protease inhibitor 2. When unfunctional, the protein cause a rare, inherited diarrhea of infancy named congenital secretory sodium diarrhea type 3 (DIAR3) or congenital sodium diarrhea (CSD).

Interesting binding partners among the singletons are: probable low affinity copper uptake protein 2, mitochondrial carrier homologue 2 (transporter of an unknown solute), calcium release-activated calcium channel protein 1, zinc transporter ZIP13 and solute carrier family 35 member B1 which is likely to be an endoplasmatic reticulum located sugar transporter (Uniprot).

Further substances, which either increase or decrease the SLC41A1-related Na⁺/Mg²⁺ exchanger activity and are thus called modulators, are given in Table 3. These substances modulate SLC41A1-related Na⁺/Mg²⁺ exchanger activity either by direct interaction with structural components of the SLC41A1 molecule or by the induction of cellular signal cascades resulting in modifications of the SLC41A1 protein. Therefore, some of these modulators are hormones or elements of cellular signal cascades. Main sites for posttranslational modifications and thus, regulation/modulation of SLC41A1-related Na⁺/Mg²⁺ exchanger activity were the following:
Glycosylation side: amino acid residue N-475
cAMP-dependent PK phosphorylation sides: S-157, S-308, S-393, T-508
PK C phosphorylation sides: T-80, T-167, T-387, S-407
Casein kinase II motif at residue S-317
Myristoylation sequence at G-143

TABLE 3

| | | Molecular | | | Effect on | |
|---|---|---|---|---|---|---|
| | | weight | Dosage | | Na⁺/Mg²⁺ | |
| Modulator | Chemical name | (g/mol) | (μM) | | exchanger | Further Effect on |
| Imipramine | 10,11-Dihydro-N,N- | 280.4 | 250 | | Inhibition | Na⁺ channel blocker |
| | dimethyl-5H-dibenz- | | | | | |
| | [b,f]azepin-5-propanamin | | | | | |
| Quinidin | (2-ethenyl-4-azabicyclo | 324.4 | 100 | | Inhibition | K⁺ channel blocker |
| | [2.2.2] oct-5-yl)-(6- | | | | | |
| | methoxyquinolin-4-yl)- | | | | | |
| | methanol | | | | | |
| DIDS | 4′,4′- | 500.48 | 100 | | Inhibition | Cl⁻ channel inhibitor, |
| | diisothiocyanatodihydrostil | | | | | Anion exchanger inhibitor |
| | bene-′,2′-disulfonic acid, | | | | | |
| | disodium salt | | | | | |
| Lidocaine | 2-(diethylamino)- | 234.34 | | | Inhibition | Na⁺ channel blocker |
| | N-(2,6-dimethylphenyl)- | | | | | |
| | acetamide | | | | | |
| KB-R7943 | 2-(2-(4-(4-nitrobenzyloxy)- | 427.50 | | | Inhibition | Na⁺/Ca²⁺ exchanger inhibitor |
| | phenyl)ethyl)isothiourea | | | | | |
| | methanesulfonate | | | | | |
| SEA0400 | 2-[4-[(2,5-difluoro- | 371.38 | | | Inhibition | Na⁺/Ca²⁺ exchanger inhibitor |
| | phenyl)methoxy]phenoxy]- | | | | | |
| | 5-ethoxyaniline | | | | | |
| Benzamil | 3,5-diamino-N-[(1E)- | 319.75 | 10 | μM | Inhibition | Na⁺/Ca²⁺ exchanger inhibitor |
| | amino(benzylamino)methyl | | | | | |
| | idene]-6-chloropyrazine-2- | | | | | |
| | carboxamide | | | | | |
| Amiloride | 3,5-diamino-6-chloro-N- | 229.63 | 5−100 | μM | Inhibition | Na⁺/H⁺ exchanger (NHE) inhibitor |
| | (diaminomethylidene)pyraz | | | | | |
| | ine-2-carbox-amide | | | | | |
| Nifedipine | 3,5-dimethyl 2,6-dimethyl- | 346.36 | | | Inhibition | Ca²⁺ channel blocker |
| | 4-(2-nitrophenyl)-1,4-di- | | | | | |
| | hydropyridine-3,5-di- | | | | | |
| | carboxylate | | | | | |
| Angiotensin II | | | 100 | nM | activation | Vasoactive agent, vasoconstriction |
| (via AT 1 | | | | | | |
| receptor) | | | | | | |
| Aldosterone | | | | | activation | |
| Glucocorticoids | Dexamethason | 392.46 | | | | |
| Catecholamines, | Norepinephrine, | | | | activation | |
| β-adrenergic | isoproterenol | | | | | |
| agonists | | | | | | |
| α₁-adrenergic | | | | | | |
| agonists | | | | | | |
| cAMP and cAMP | 8-Cl-cAMP; dibutyryl- | | 100 | μM | activation | |
| analogues | cAMP | | | | | |
| | Rp-cAMP | | | | inhibition | |
| Forskolin | [(3R,4aR,5S,6S,6aS,10S,1 | 410.50 | | | activation | |
| | 0aR,10bS)-3-ethenyl- | | | | | |
| | 6,10,10b-trihydroxy- | | | | | |
| | 3,4a,7,7,10a-pentamethyl- | | | | | |
| | 1-oxo-5,6,6a,8,9,10- | | | | | |
| | hexahydro-2H- | | | | | |
| | benzo[f]chromen-5-yl] | | | | | |
| | acetate | | | | | |
| Cardiotonic | Ouabain, rostafuroxin, | | | | inhibition | |
| steroids | bufalin, oleandrin, | | | | | |
| (glycosides) | marinobufagenin, | | | | | |
| | UNBS-1450 | | | | | |
| Glucagon | | | | | activation | |
| Insulin | | | 200 | μU/ml | inhibition | |
| (via insulin | | | 400 | μU/ml | | |
| receptor) | | | | | | |
| Genistein | 5,7-dihydroxy-3-(4- | 270.23 | 10 | μM | inhibition | Tyrosine kinase inhibitor |
| | hydroxyphenyl)chromen-4- | | | | | |
| | one | | | | | |
| Mitogens | Bombesin, epidermal | | | | inhibition | |
| | growth factor (EGF) | | | | | |
| Taurine | 2-aminoethanesulfonic | | 10−20 | mM | inhibition | |
| | acid | | | | | |
| Organic | | | | | | |
| polycations | | | | | | |
| Spermine, | N′,N′-bis(3-aminopropyl)- | | | | | |
| spermidine | butane-1,4-diamine, N-(3- | | | | | |
| | aminopropyl)butane-1,4- | | | | | |
| | diamine | | | | | |
| PhTX-343 | S-N-[3-[[4-[(3- | | | | | Derivative of the naturally occuring |
| | Aminopropyl)amino]butyl]- | | | | | polyamine toxin philantotoxin |
| | amino]propyl]-4-hydroxy- | | | | | |
| | alpha-[(1-oxobutyl)amino]- | | | | | |
| | benzenepropanamide tris- | | | | | |
| | trifluoroacetate (Organic | | | | | |
| | polycation) | | | | | |
| Agmatine | 2-(4-aminobutyl)guanidine | 130.19 | | | | Polyamine in neurons |
| L-NMMA | NG-monomethyl-L-arginine | | 100 | μM | inhibition | NO inhibitor |
| ODQ | 1H-[1,2,4]oxadiazolo[4,3- | | 10 | μM | inhibition | inhibitor of NO-sensitive guanylate cyclase |
| | α]quinoxaline-1 | | | | | |
| NOR1 | (E)-4-methyl-2-[(E)- | | 100 | μM | activation | NO donor |
| | hydoxyimino]-5-nitro-6- | | | | | |
| | methoxy-3-hexenamide | | | | | |
| Calphostin C | 1-[3,10-dihydroxy-12-[2-(4- | 790.76 | 1 | μM | inhibition | PK C inhibitor |
| | hydroxyphenoxy)- | | | | | |
| | carbonyloxypropyl]- | | | | | |
| | 2,6,7,11-tetramethoxy-4,9- | | | | | |
| | dioxoperylen-1-yl]propan- | | | | | |
| | 2-yl benzoate | | | | | |
| Phorbol 12,13- | | 504.61 | 1 | μM | activation | PK C activator |
| dibutyrate | | | | | | |
| cGMP and | 8-bromo-cyclic GMP | | 100 | μM | activation | |
| cGMP analogues | | | | | | |
| PGE₂ | (Z)-7-[(1R,2R,3R)-3- | 352.47 | 100 | nM | activation | |
| | hydroxy-2-[(E,3S)-3- | | | | | |
| | hydroxyoct-1-enyl]-5- | | | | | |
| | oxocyclopentyl]hept-5- | | | | | |
| | enoic acid | | | | | |
| Indomethacin | 2-[1-(4-chlorobenzoyl)-5- | 357.79 | 10 | μM | activation | Arachidonic acid metabolism inhibitor |
| | methoxy-2-methylindol-3- | | | | | |
| | yl]acetic acid | | | | | |
| Arachidonic acid | (5Z,8Z,11Z,14Z)-icosa- | 304.47 | 30 | μM | activation | |
| | 5,8,11,14-tetraenoic acid | | | | | |

Particularly imipramine, quinidin, cyclic adenosine monophosphate (cAMP) analogues, β-adrenergic and α₁-adrenergic agonists and the hormones angiotensin II and insulin showed strong modulating effects. It could be demonstrated that replacement of extracellular Na⁺ with N-methyl-D-glutamine (NMDG) or application of the unspecific Na⁺/Mg²⁺ exchanger inhibitors imipramine and quinidin strongly decrease SLC41A1-mediated efflux. Also, it was found that SLC41A1-mediated Mg²⁺ efflux is stimulated by db-cyclic adenosine monophosphate (db-cAMP), adrenergic receptor agonists and the hormone angiotensin II. In contrast, an inhibitory effect was observed after application of insulin.

In view of the importance of the Na⁺/Mg²⁺ exchanger activity to cell metabolism, viability and proliferation, we believe that SLC41A1 represent an ideal target for designing and/or identifying molecules that block or stimulate these processes.

Therefore, the above mentioned modulators (Table 3), especially imipramine, quinidin, DIDS, lidocaine, nifedipine, benzamil, KB-R7943, SEA0400, amiloride, agonists or antagonists of receptors for glucocorticoids, aldosterone, angiotensin II, insulin, glucagon, PGE₂ and catecholamines (β-adrenergic and/or α1-adrenergic agents), cAMP or cGMP analogues, taurine, genistein, calphostin C, phorbol 12,13-dibutyrate, L-NMMA, NOR1, ODQ, forskolin, indomethacin, arachidonic acid, spermine, spermidine, PhTX-343, agmatine, cardiotonic steroids (e.g. ouabain, rostafuroxin, bufalin, oleandrin, marinobufagenin, UNBS-1450) and of mitogens (e.g. bombesin, EGF), more preferably imipramine, quinidin, CAMP and its analogues, adrenergic agonists and molecules that mimic the effects of angiotensin II and of insulin, can be used to modulate the function, metabolism, proliferation and growth of Na⁺/Mg²⁺ exchanger expressing cells. The above named modulators are also intended for the use as therapeutic agents.

The above-mentioned antibodies, protein binding partners and the above named modulators or their analogues or agents acting as agonists or antagonists of these modulators are believed to be useful for treating patients having conditions that are characterized by increased or decreased Mg²⁺ efflux via their Na⁺/Mg²⁺ exchanger expressing cells. Such conditions of disturbed or dysregulated Na⁺/Mg²⁺ exchanger activity have been described in various disease complexes such as cardiovascular disorders, metabolic and endocrine dysfunctions, neurologic diseases, cancer, pre-eclampsia, eclampsia, fetal growth retardation and premature labor.

Special diseases out of these disease complexes, which the present invention is focused on, are listed in Table 4. Diseases, on which the invention is preferably focused on, are hypertension, ischemic heart disease, myocardial infarction, cardio-vascular dysfunctions in relation to diabetes mellitus, pre-eclampsia, eclampsia, diabetes mellitus (type II), non-insulin-dependent diabetes, cystic fibrosis, morbus parkinson, stroke, prostate cancer as well as aggressive behaviour or high stress sensitivity. These diseases are marked in Table 4 (bold).

TABLE 4

| | | Role of | | |
|---|---|---|---|---|
| Disease | | Na⁺/Mg²⁺ | Pathogenic | Relevancy/strength |
| complex | Disease | exchanger | factors | of evidence |
| Cardiovascular | Hypertension | hyperactivity | Potentiation of | ***** (5) |
| | (high blood | | platelet | |
| | pressure) | | aggregation, | |
| | ischemic heart | | Loss of Mg²⁺ | |
| | disease, | | from heart | |
| | myocardial | | muscle cells, | |
| | infarction | | hepatocytes, | |
| | congestive heart | | platelets and | |
| | failure | | smooth vascular | |
| | atherosclerosis | | muscle cells, | |
| | cardiac arrhythmias | | Increased | |
| | sudden cardiac | | sensitivity to | |
| | death | | vasoconstrictors | |
| | Cardiovascular | | | |
| | dysfunction in | | | |
| | relation to | | | |
| | diabetes mellitus | | | |
| | Pre-eclampsia, | | | |
| | Eclampsia | | | |
| | (strongly related to | | | |
| | pregnancy) | | | |
| Metabolism | Diabetes mellitus | hyperactivity | | **** (4) |
| Endocrine | (type I) | | | |
| dysfunction | Non-insulin- | | | |
| | dependent | | | |
| | diabetes | | | |
| | (type II) | | | |
| | Hyoeraldosteronism | | | |
| | Hyperthyroidism/ | | Increase/loss of | |
| | hypothyroidism | | Mg²⁺ in/from | |
| | | | heart muscle | |
| | | | cells | |
| | Mukoviszidose | hyperactivity | | |
| | Cystic fibrosis | | | |
| Neurology/ | Parkinson | | | ** (2) |
| behaviour | disease, Morbus | | | |
| | Parkinson | | | |
| | Aggressive | hyperactivity | | |
| | behaviour, high | | | |
| | stress sensitivity | | | |
| | stroke | | | |
| Fetal growth | | | | ** (2) |
| retardation, | | | | |
| premature | | | | |
| labor | | | | |
| cancer | Prostate cancer | Upregulated | | ** (2) |
| | | hyperactivity | | |

In Table 4, the diseases are listed from top to bottom according to the present evidence that their pathogenesis is related to an increased or decreased Mg²⁺ efflux via Na⁺/Mg²⁺ exchanger expressing cells. The strength of evidence is indicated by stars in the last column. Cardiovascular diseases have the strongest evidence that they are related to the Na⁺/Mg²⁺ exchanger. Thus, the treatment of cardiovascular diseases is the most relevant application for the present invention, i.e. for the antibodies, the special protein binding partners as outlined above or for the specific modulators outlined above.

The nucleotide sequence of SEQ ID NO 3 (cDNA) is of special use for the gene therapy of these diseases, i.e. for the treatment of a disease from the group of cardiovascular diseases, metabolic or endocrine dysfunctions, neurological diseases, cancer, pre-eclampsia, eclampsia, fetal growth retardation and premature labor, preferably for the treatment of hypertension, ischemic heart disease, myocardial infarction, cardiac dysfunction in relation to diabetes mellitus, pre-eclampsia, eclampsia, diabetes mellitus (type I), non-insulin-dependent diabetes, mukoviszidose, cystic fibrosis (type II), parkinson disease, morbus parkinson, stroke, prostate cancer as well as aggressive behaviour or high stress sensitivity. The same applies to a fragment of SEQ ID NO 3, in which one or more nucleotides are replaced, deleted or added and which still has an identity on the nucleotide level of at least 90%.

Another aspect of the present invention is a model based on a non-human mammal, preferably a mouse, having an inducible conditional system leading to a functional knock out of SLC41A1, i.e. a genetically modified conditional knock out non-human mammal, which is characterized in that the SCL41A1 gene is inducible conditionally knockable (knock out), i.e. it can be knocked-out in a time (e.g., at embryonic, young or adult stage of development) or tissue-specific (particular organ or cell type) manner. In the following, the mouse is referred to because this is the preferred non-human mammal according to the present invention.

The mouse model was developed on the basis of the gene sequence of SLC41A1 (SEQ ID NO 3, GenBank Accession No. NM_{—}173854).

A SLC41A1 inducible conditional KO mouse was developed by homologous recombination using 129 embryonic stem cells. This mouse line was then crossed with Cre recombinase (CR) or ligand-dependent Cre recombinase (LCR) expressing mice for the generation of tissue specific or time dependent targeted inactivation of murine SLC41A1.

LCR are fusion proteins between Cre and the ligand-binding domains (LBDs) of steroid receptors. The LBD has been mutated so that it does not respond to its natural ligand yet binds a synthetic ligand. As an example may serve tamoxifen-activated CreERT recombinase (modified pacman), a fusion of Cre with a mutated estrogen receptor (ER) LBD, that responds specifically to the synthetic drug 4-hydroxytamoxifen (OHT) but not to β-estradiol. In the absence of OHT, the recombinase is located in the cytoplasm. Binding of OHT to the LBD results in the translocation of the recombinase into the nucleus where it can recombine its loxP substrates (Feil R., 2007). The LCR system is widely used for the generation of inducible conditional KO mice in experiments with time dependent settings but also in those with demand for tissue specificity. Mouse lines expressing CreERT2 recombinase in tissue specific manner are listed below and the newly developed conditional KO SLC41A1 mouse can be bread with one of the available CreERT2 mouse lines in dependence of specific experimental needs.

Examples of mouse lines expressing the CreERT2 recombinase in specific tissues:

Bone (osteoblasts Col1a1-CreERT2 tg Collagen 1 α1 chain and odontoblasts)
Endothelium (Tie2-CreERT2 tg Tie2 receptor tyrosine kinase)

### Epithelium:

Intestinal epithelium Vil-CreERT2 tg Villin el
Internal epithelial organs K18-CreERT2 tg Keratin 18
Renal epithelium KspCad-CreERT2 tg Kidney-specific cadherin Lantinga-van
Fat tissue (adipocytes) aP2-CreERT2 tg Adipocyte fatty acid binding protein
Liver (hepatocytes) SA-CreERT2 ki Serum albumin

### Nervous System:

Astrocytes GFAP-CreERT2 tg Glial fibrillary acidic protein
GLAST-CreERT2 ki Astrocyte-specific glutamate transporter
Neural stem cells Nes-CreERT2 tg Nestin
Schwann cells and PLP-CreERT2 tg Proteolipid protein
oligodenrocytes
Schwann cells P0Cx-CreERT2 tg P0 fused to connexin 32

### Skin:

### Keratinocytes K5-CreERT2 tg Keratin 5

### K14-CreERT2 tg Keratin 14

### Melanocytes Tyr-CreERT2 tg Tyrosinase

### Tyr-CreERT2 tg Tyrosinase

### Widespread Rosa26-CreERT2 ki Rosa26.

### Muscle:

Skeletal muscle HAS-CreERT2 tg (PAC) Skeletal muscle α-actin
Smooth muscle SM-CreERT2 ki SM22α

The transgenic conditional knock out mouse is characterized in that the genome comprises a construct, which allows conditional knock out of the SCL41A1. The term “construct” refers to a polynucleotide, which was exogenously inserted into the genom of the animal. The animal can be heterozygous or homozygous but is preferably homozygous with respect to the knock out gene (^{-/-}). The construct comprises the SCL41A1 gene of SEQ ID NO 4 (murine gene) flanked by two loxP DNA sequence elements (loxP sites), i.e. they are preferably situated 3′ and 5′ of the SCL41A1 gene.

Isolated cells from various tissues allow further investigation of the function of SCL41A1. Thus, another aspect of the invention is a transgenic inducibleconditional knock out cell or cell line. Cells can be taken from the transgenic inducible conditional knock out non-human mammal or can originate from separate cell lines.

If cells from the transgenic animal are concerned, it is preferred to use primary cell cultures (primocultures) from the heart (cardiomyocytes), from the vasculature (e.g. aorta), from the liver (hepatocytes) or from the brain or CNS/microglial cells of these animals.

If cells or cell lines of other origin are concerned, it is preferred to use stable human cell lines, more preferably stable human embryonic kidney cell line (ACC-305/DSMZ) with inducible conditionally knocked out SLC41A1 gene, HEK293 (hSLC41A1^{-/-}) or stable human B-cell precursor leukemia cell line (ACC-128/DSMZ) with inducible conditionally knocked out SLC41A1 gene, NALM-6 (hSLC41A1^{-/-}). Cell lines will be generated according to the technique described by Shibahara Kei-ichi et al.

To generate inducible conditionally knocked out SLC41A1 cells or cell lines, Mammalian vectors, which comprise mutated forms of human SLC41A1 are transferred or electroporated into these primocultured cells of or into cells of the stable human cell lines, resulting in cells having in their genome mammalian vectors with a mutated human SLC41A1. The mutated forms of human SLC41A1 are as described above with respect to the mutation libraries, i.e. each of these mutated sequences can be present in the vector.

### DESCRIPTION OF THE FIGURES

FIG. 1 shows Mg²⁺ Efflux in dependence of the Mg²⁺ concentration of the loading medium.
A: Representative original recordings of [Mg²⁺]ᵢ changes after re-suspension of −tet and +tet HEK293-(SLC41A1) cells in Mg²⁺-free, Na⁺-containing medium. Before measurements cells had been loaded with Mg²⁺ by incubating them in a high (10 mM)-Mg²⁺ solution for 20 min.
B: Magnitude of mean [Mg²⁺]ᵢ changes for −tet and +tet HEK293-(SLC41A1) cells pre-loaded in solutions containing 0, 2, 5 or 10 mM Mg²⁺. N=10 single experiments for each Mg²⁺ concentration;
P<0.01; ** P<0.001.

FIG. 2 shows the effect of complete substitution of extracellular Na⁺ by NMDG on SLC41A1-dependent Mg²⁺ efflux. A: Representative original recordings of [Mg²⁺]ᵢ changes after re-suspension of −tet and +tet HEK293-(SLC41A1) cells in Mg²⁺-free, Na⁺-containing or in Mg²⁺- and Na⁺-free medium. Before measurements cells had been loaded with Mg²⁺ by incubating them in a high (10 mM)-Mg²⁺ solution for 20 min. B: Effect of Na⁺ substitution on [Mg²⁺]ᵢ of −tet and +tet HEK 293-(SLC41A1) cells before and after a 20 min incubation in completely Mg²⁺-free medium. N=9 single experiments per condition; ** P<0.001. C: Effect of Na⁺ substitution on SLC41A1-dependent Mg²⁺ extrusion measured as decrease of [Mg²⁺]ᵢ. N=9; P<0.001.Mg²⁺

FIG. 3: shows the effect of imipramine and of quinidin on SLC41A1-dependent Mg²⁺ efflux. A: Representative original recordings of [Mg²⁺]ᵢ changes after re-suspension of +tet HEK293-(SLC41A1) cells in Mg²⁺-free, Na⁺-containing media with or without imipramine (250 μM). Before measurements cells were loaded with Mg²⁺ by incubating them in a high (10 mM)-Mg²⁺ solution for 20 min. B: Summary of the results obtained from 8 single measurements per condition. ** P<0.001. C: Summary of the effect of quinidin (100 μM) on Mg²⁺]ᵢ changes observed after re-suspension of +tet HEK293-(SLC41A1) cells in Mg²⁺-free, Na⁺-containing media. Before measurements cells were loaded with Mg²⁺ by incubating them in a high (10 mM)-Mg²⁺ solution for 20 min. N=3 single measurements per condition.

FIG. 4: shows the effect of dB-cAMP on [Mg²⁺]ᵢ decrease in +tet HEK293-(SLC41A1) cells The [Mg²⁺]ᵢ decreased observed without and with dB-cAMP (100 μM) is shown. Bars represent means±SE from 6 (0 mM-loading), to 10 (2 mM and 10 mM-loadingl) single measurements.

FIG. 5: Original traces showing the effect of imipramine (250 μM) and of dB-cAMP (100 μM) on the proliferation of HEK293-(SLC41A1) cells. The respective agents are added 24 hours after seeding. Increase or decrease of the cell index reflects a higher or lower proliferation rate, respectively. Red curve: control; blue curve: imipramine; green curve=dB-cAMP.

FIG. 6: Effect of insulin on the SLC41A1-dependent Mg²⁺ efflux in −tet and +tet HEK293-(SLC41A1) cells. [Mg²⁺]ᵢ changes after re-suspension of −tet and +tet HEK293-(SLC41A1) cells in Mg²⁺-free, Na⁺-containing media without or with insulin (200 μU/ml and 400 μU/ml) are shown. Before measurements cells were loaded with Mg²⁺ by incubating them in a high (10 mM)-Mg²⁺ solution for 20 min. N=3 single measurements per condition.

Below, the invention is outlined in more detail by means of example.

### EXAMPLES

### Material and Methods

### HEK293-(FLAG-SLC41A1) Cell Line; Growth Media and Culture Conditions

Preparation (cloning protocol) and culture conditions of tetracycline-(tet)-inducible HEK293-(FLAG-SLC41A1) cell line were previously described by Kolisek et al. [2008].

### Western Blot Analysis

Non-induced (−tet) and induced (+tet, 15-18 h) HEK293-(FLAG-SLC41A1) cells (107 cells ml⁻¹) were lysed for 30 min at 4° C. in Tris buffer (pH 7.5) containing 1% Triton X-100 (Bio-Rad) and protease inhibitors (Perbio-ThermoFisher). Total lysate proteins had been resolved by 10% SDS-PAGE, transferred to polyvinylidene difluoride membranes, and immunodecorated with anti-FLAG antibody coupled to HRP (Invitrogen), or with antibody to -actin (AbCam, Cambridge, UK) conjugated to GAM HRPlinked antibody (Jackson ImmunoResearch Laboratories, West Grove, Pa.). The same samples were immunoprecipitated by M2 anti-FLAG (Sigma) or isotype control, resolved by 10% SDS-PAGE, and transferred to polyvinylidene difluoride membranes. The membrane was immunoblotted with M2 anti-FLAG (Sigma) and GAM-_-HRP (SBA, Birmingham, Ala.). Membranes were developed by enhanced chemical luminescence (ECL) (Amersham Biosciences).

Determination of Free Intracellular Mg²⁺ in +tet and −tet HEK293-(SLC41A1) Cells by mag-fura 2 FF-Spectrofluorometry

The uninduced (−tet) and induced (+tet) HEK293-(SLC41A1) cells were rinsed twice with ice-cold, completely divalent-free Dulbecco's phosphate-buffered saline (DPBS), detached by use of Hytase (Perbio Science, Bonn, Germany), centrifuged, washed twice in completely Ca²⁺- and Mg²⁺-free Hank's balanced solution (CMF-HBS, PAN Biotech, Aidenbach, Germany) complemented with 10 mmol.l⁻¹ HEPES and 1.36 mmol.l⁻¹ L-glutamine (CMF-HBS+) and finally re-suspended in the same solution. Loading of cells with 7.5 μmol.l⁻¹ mag-fura 2 AM (Molecular Probes, Eugene, Oreg. USA) was performed for 30 minutes at 37° C. in the presence of loading facilitator Pluronic F-127 (Molecular probes). After being washed in CMF-HBS+, cells were incubated for a further 30 minutes at 37° C. to allow for complete de-esterification of the fluorescence probe, washed twice in CMF-HBS+ to remove extracellular mag-fura 2 and stored in CMF-HBS supplemented with 10 mmol.l⁻¹ HEPES, 5 mmol.l⁻¹ glucose and 0.4 mmol.l⁻¹ Mg²⁺ until being Mg²⁺-loaded before measurements of the Mg²⁺ efflux capacity. The latter was determined as the intracellular Mg²⁺ concentration ([Mg²⁺]ᵢ) decrease over a 20-min period. Directly before the efflux experiments, −tet and +tet HEK293-(SLC41A1) were Mg-loaded by a 20-min incubation in CMF-HBS+ containing 0, 2, 5 or 10 mmol.l⁻¹ Mg. An extracellular Mg²⁺ concentration ([Mg²⁺]ₑ) of 10 mmol.l⁻¹ was selected for all further experiments. After loading, the remaining extracellular Mg²⁺ was removed by washing the cells twice in CMF-HBS+ and, after re-suspending the cells in CMF-HBS+, measurements were started. To determine the influence of varying extracellular [Na⁺], measuring solutions were prepared by diluting the CMF-HBS+ containing 145 mM Na⁺ with appropriate volumes of a completely Na⁺- and Mg²⁺-free PBS. To investigate the effect of HCO₃⁻ on Mg²⁺ efflux, completely Ca²⁺- and Mg²⁺-free medium 199 completed with 10 mmol.l⁻¹ HEPES and 1.36 mmol.l⁻¹ L-glutamine (CMF-M199+) with Hank's (0.35 g/l NaHCO₃) or Earl's (2.2 g/l NaHCO₃) salts were used during Mg-loading and measurements. To ensure a stable pH value, the high-HCO₃⁻ media were pre-equilibrated with 95% air-5% CO₂.

Measurements were made at 37° C. in 3 ml cuvettes containing 2 ml cell suspension (CMF-HBS+ or CMF-M199+ with a cytocrit of 10%) under stirring. [Mg²⁺]ᵢ was determined by measuring the fluorescence of the probe-loaded cells in a spectrofluorometer (LS50-B, PerkinElmer Life Science, Wellesley, Mass. USA) by using the fast filter accessory, which allowed fluorescence to be measured at 20-ms intervals with excitation at 340 and 380 nm, and emission at 515 nm. [Mg²⁺]ᵢ values were calculated from the 340/380-nm ratio according to the formula of Grynkiewicz et al. (1981) by using the software FL WinLab version 4.0 (PerkinElmer Life Science). A dissociation constant of 1.5 mmol.l⁻¹ for the mag-fura 2/Mg²⁺ complex was used for calculations; minimum (Rₘᵢₙ) and maximum (Rₘₐₓ) ratios were determined at the end of each experiment by using digitonin. Rₘₐₓ was found by the addition of 25 mmol.l⁻¹ MgCl₂ in the absence of Ca²⁺, whereas Rₘᵢₙ was obtained by addition of 50 mmol.l⁻¹ EDTA, pH 7.2, to remove all Mg²⁺ from the solution. For data evaluation, 10-second data sets each were averaged at the beginning of the measurement and then always after 50 seconds. The final [Mg²⁺]ᵢ was determined as the mean [Mg²⁺]ᵢ of the last 10 seconds of the measurement. Thus, for the calculation of any given [Mg²⁺]ᵢ, 500 data points were used. If not otherwise stated, data are presented as means±standard error (SE).

Table 5
List of special media

| Name | Remarks | Producer |
|---|---|---|
| —Cl⁻, —Mg²⁺, —Ca²⁺ | Cl⁻, Mg²⁺, Ca²⁺ free medium | |
| Hank's medium | (Cl⁻ has been substituted with | |
| | glutamate) | |
| Completely Mg²⁺— and | Without Ca²⁺/Mg²⁺, Na⁺ | No. LZ 1825, Biochrom, Berlin, |
| Na⁺— free | substituted by N-methyl-D- | Germany |
| PBS | glucamine (NMDG) | |
| Ca²⁺— and Mg²⁺— free | Without Ca²⁺/Mg²⁺ and phenol | No. FZ 0635, Biochrom, Berlin, |
| medium 199 Hanks | red | Germany |
| Ca²⁺— and Mg²⁺— free | Without Ca²⁺/Mg²⁺ and phenol | No. FZ 0615, Biochrom, Berlin, |
| medium 199 Earl | red | Germany |

TABLE 6
List of inhibitors

| | | | Final | | |
|---|---|---|---|---|---|
| | | MW | concentration | | |
| Name | Formula | (g.mol-1) | (mol.l-1) | Producer | Inhibitor of: |
| Imipramine | 10,11-Dihydro-N,N-dimethyl- | 280.4 | 250.10-6 | ICN | A, F |
| | 5H-dibenz-[b,f]azepin-5- | | | Biomedicals, | |
| | propanamin | | | Irvine, CA, | |
| | | | | USA | |
| Quinidin | (2-ethenyl-4-azabicyclo | 324.4 | 100.10-6 | | A, E |
| | [2.2.2] oct-5-yl)-(6- | | | | |
| | methoxyquinolin-4-yl)- | | | | |
| | methanol | | | | |
| Verapamil | 5-[N-(3,4- | 491.06 | 100.10-6 | Sigma- | D |
| | dimethoxyphenylethyl)methy | | | Aldrich, | |
| | lamino]-2-(3,4- | | | St. Louis, | |
| | dimethoxyphenyl)-2- | | | MO USA | |
| | isopropylvaleronitrile | | | | |
| | hydrochloride | | | | |
| Niflumic | 2-[3- | 282.22 | 100.10-6 | Sigma- | C |
| acid | (trifluoromethyl)anilino]nicoti | | | Aldrich, | |
| | nic acid, 2-(α,α,α-trifluoro-m- | | | St. Louis, | |
| | toluidino)nicotinic acid | | | MO USA | |
| H₂DIDS | 4,4′- | 500.48 | 100.10-6 | Molecular | B, C |
| | diisothiocyanatodihydrostil- | | | Probes, | |
| | bene-2,2′-disulfonic acid, | | | Eugene, | |
| | disodium salt | | | OR USA | |
| NBBP | 5-nitro-2-(3-phenyl | 300.31 | 100.10-6 | Sigma- | C |
| | propylamino)benzoic acid | | | Aldrich, | |
| | | | | St. Louis, | |
| | | | | MO USA | |

A: inhibitor of Na⁺ channels and of Na⁺-dependent Mg²⁺ efflux system
B: wide range chloride (anion) transporter inhibitor
C: Cl channel inhibitor
D: L-type Ca²⁺ channels inhibitor
E: K⁺ channel inhibitor
F: Na⁺ channel inhibitor

### Results

### [Mg²⁺]ᵢ of −tet and +tet HEK293-(SLC41A1) Cells.

The basal [Mg²⁺]ᵢ of Mag-Fura 2-loaded −tet and +tet (15 hrs) HEK293-(SLC41A1) cells was measured in completely Mg-free but Na-containing medium after a 20-min pre-incubation in the same solution (table 7). Compared to −tet cells (0.26±0.05 mM), tetracycline-induced HEK293-(SLC41A1) cells had a 62% lower [Mg²⁺]ᵢ (0.10±0.02 mM). In agreement with our former results [Kolisek et al., 2008] these data point to an increased efflux capacity of HEK293 cells over-expressing SLC41A1. To proof this, the efflux capacity of differently Mg-loaded −tet and +tet cells was determined.

### [Mg²⁺]ᵢ Change in Differently Mg-Loaded −tet and +tet HEK293-(SLC41A1) Cells.

As it is well known that the [Mg²⁺]ᵢ influences the Mg²⁺ efflux activity, the experiments were designed to support SLC41A1-related Mg²⁺ release by performing all preparation and storage procedures (with exception of probe-loading) before the actual measurements in Mg-containing solutions. In addition, directly before the efflux experiments, −tet and +tet HEK293-(SLC41A1) cells were differently Mg-loaded by a 20-min incubation in media containing 2, 5 or 10 mmol.l⁻¹ Mg. Afterwards, the Mg²⁺ efflux capacity was determined as [Mg²⁺]ᵢ decrease measured over a 20-mins period in completely Mg-free media. In table 6, [ Mg²⁺]ᵢ values determined at the beginning and end of the measuring period are given for all conditions. It can be seen that −tet HEK293-(SLC41A1) cells had a stable [Mg²⁺]ᵢ of 0.25±0.02 mM and no increase or decrease was observed after Mg-loading or incubation in completely Mg-free medium. In contrast, pre-incubation with increasing extracellular Mg²⁺ concentrations induced a ≈3- to 4-fold elevation of the [Mg²⁺]ᵢ in +tet HEK293-(SLC41A1) cells (table 7). When re-suspended in absolutely Mg²⁺-free media these cells show a continuous [Mg²⁺]ᵢ decrease (FIG. 1A) reflecting Mg²⁺ release from the cells. Depending on loading conditions (2, 5 or 10 mM extracellular Mg) this Mg²⁺ efflux results in an 48%, 53% and 65% reduction of the endpoint [Mg²⁺]ᵢ compared to the respective starting level (Table 7). The results for −tet and +tet (15 hrs) HEK293-(SLC41A1) cells are summarized in FIG. 1B. In comparison to non-induced −tet HEK293-(SLC41A1) cells, a significant Mg²⁺ extrusion occurred when +tet HEK293-(SLC41A1) cells pre-loaded with 5 or 10 mM Mg²⁺ were suspended in Mg-free medium. The [Mg²⁺]ᵢ decrease during the measuring period amounted to 133±33, 224±51 and 257±44 μmol.l⁻¹ per 20 mins after pre-loading with 2, 5 and 10 mM of Mg, respectively (FIG. 1B).

TABLE 7
[Mg²⁺]ᵢ (mmol.l⁻¹) of non-induced (−tet) and induced (+tet) HEK293-(SLC41A1) cells
measured after loading with various [Mg²⁺]ₑ.

| [Mg²⁺]ₑ | −tet (control) | | +tet | |
|---|---|---|---|---|
| mM | [Mg²⁺]ᵢ at start | [Mg²⁺]ᵢ at end | [Mg²⁺]ᵢ at start | [Mg²⁺]ᵢ at end |
| 0 | 0.24 ± 0.07 | 0.28 ± 0.07 | 0.10 ± 0.02 | 0.10 ± 0.02^{A} |
| 2 | 0.23 ± 0.06 | 0.20 ± 0.06 | 0.27 ± 0.06 | 0.14 ± 0.04 |
| 5 | 0.28 ± 0.07 | 0.27 ± 0.05 | 0.43 ± 0.08^{**} | 0.20 ± 0.04^{a} |
| 10 | 0.26 ± 0.05 | 0.27 ± 0.05 | 0.40 ± 0.04^{**} | 0.14 ± 0.02^{b} |

[Mg²⁺]ᵢ values achieved after 20 min in loading solutions with various [Mg²⁺]ₑ of 0, 2, 5 or 10 mM ([Mg²⁺]ᵢ at start) and after 20 min in complete Mg-free media ([Mg²⁺]ᵢ at end) are given. Data are presented as means ± SE of 8 to 12 single experiments. A P < 0.05 vs. control (−tet cells); ^{**} P < 0.001 vs. non-loaded +tet HEK293-(SLC41A1) cells ([Mg²⁺]ₑ = 0 mM); a P < 0.05 vs. control ([Mg²⁺]ᵢ of +tet cells at start), b < 0.001 vs. control ([Mg²⁺]ᵢ of +tet cells at start)

Until now, none of the postulated Na⁺- or anion-dependent Mg²⁺ efflux systems have been identified at a molecular level. Therefore, the next step was the performance of experiments to define the transport characteristics of SLC41A1-related Mg²⁺ extrusion.

Mg²⁺ Efflux from hSLC41A1 Depends on Extracellular Na⁺ and is Inhibited by the Unspecific Na⁺/Mg²⁺ Exchanger Inhibitors Imipramine and Quinidine.

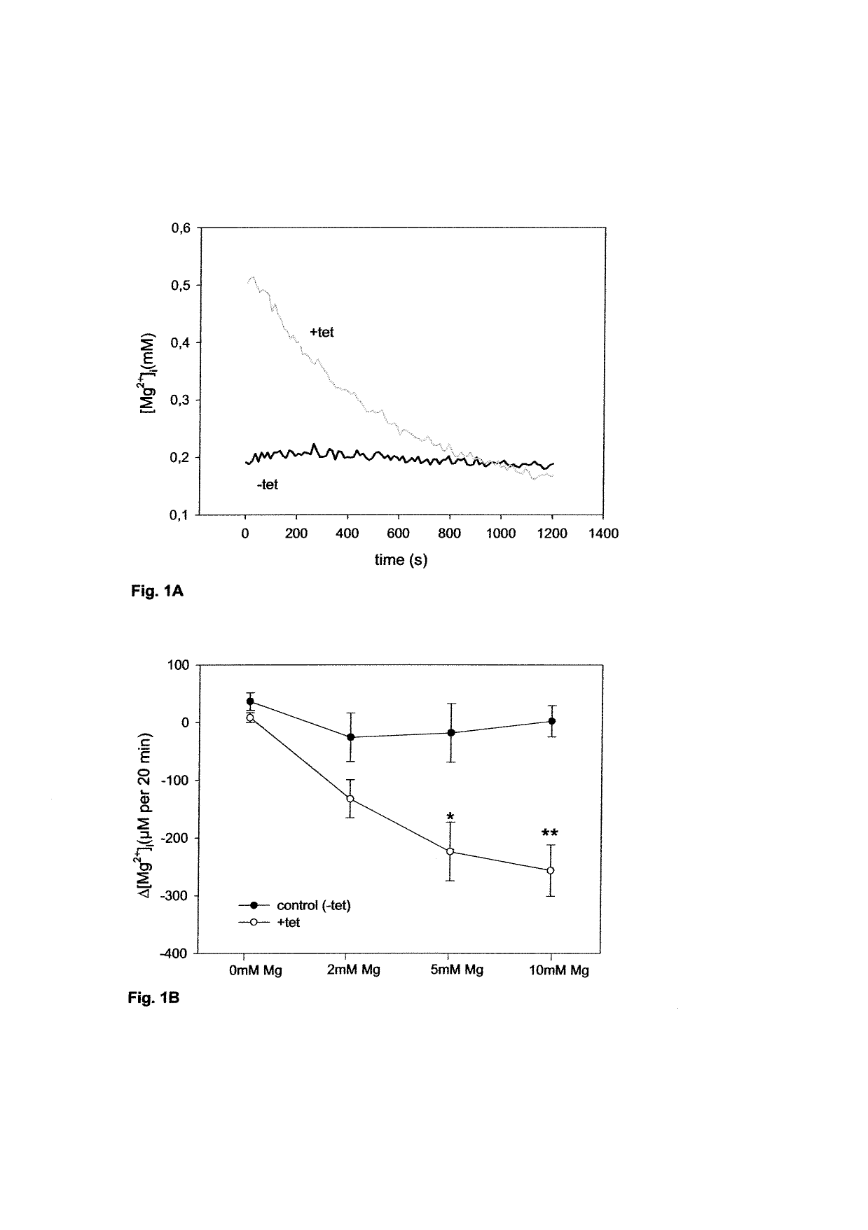

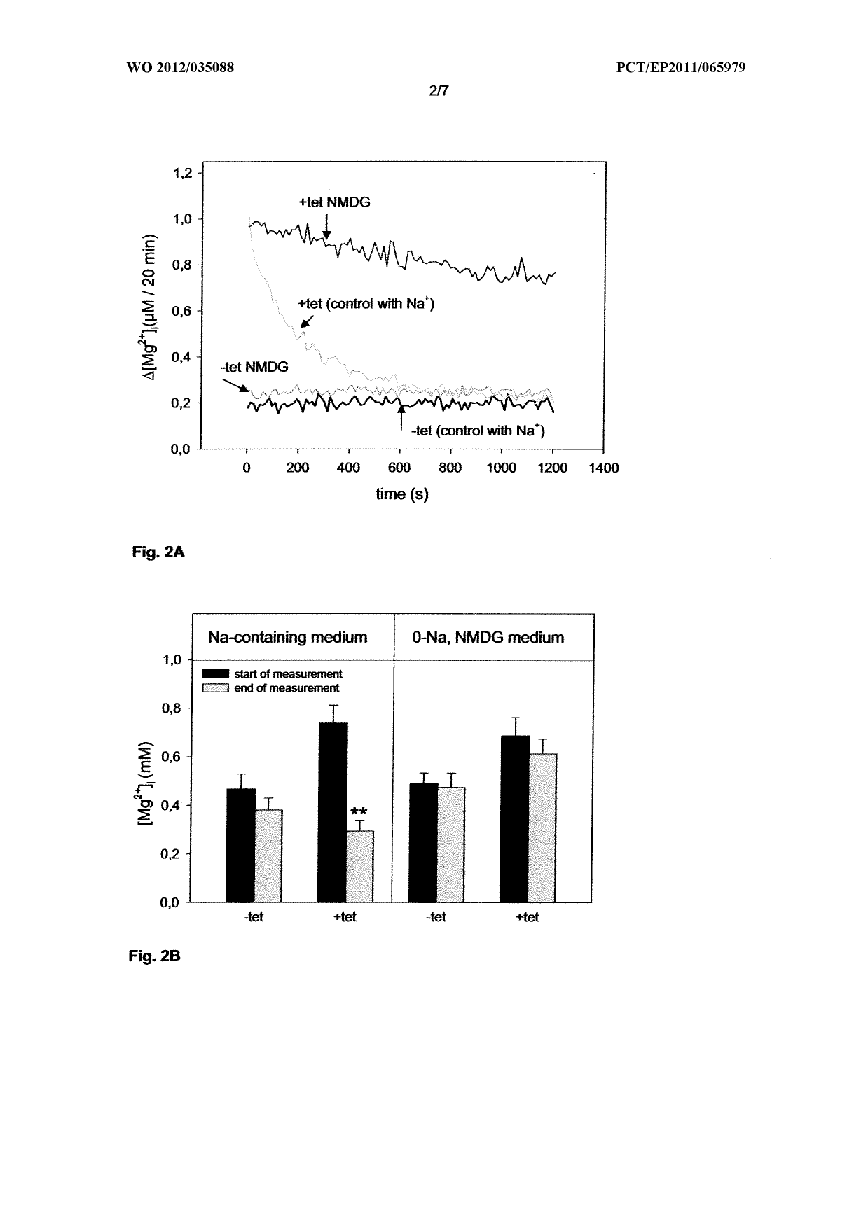

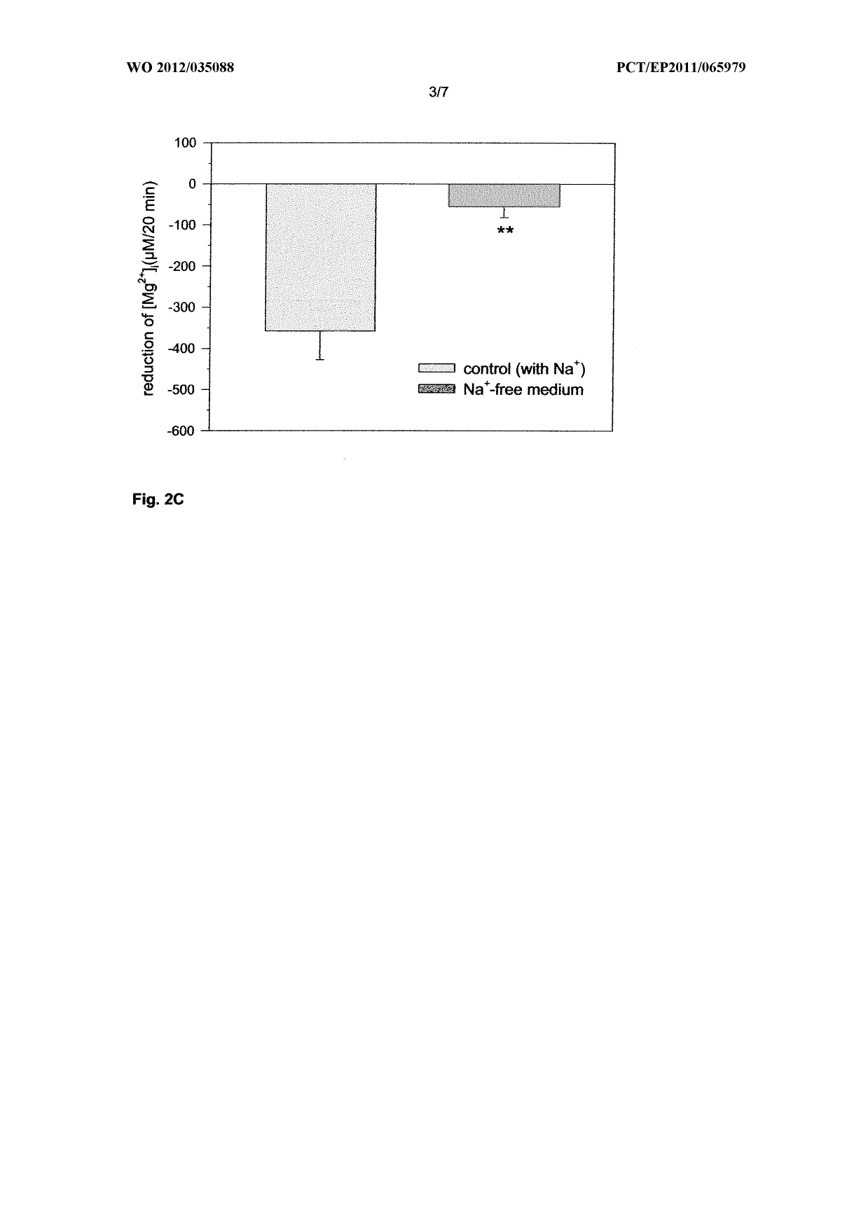

First, the effect of the extracellular [Na⁺] on the Mg²⁺ extrusion was tested with Mg-loaded (10 mM) −tet and +tet HEK293-(SLC41A1) cells. The latter were suspended in absolutely Mg-free media containing 145 mM Na⁺ or in the same medium but all Na⁺ iso-osmotically replaced by the organic non-permeant cation NMDG and the [Mg²⁺]ᵢ was measured over a 20-minute period (FIG. 2A). In −tet control cells a steady [Mg²⁺]ᵢ of 0.45±0.03 mM was determined. In contrast, the [Mg²⁺]ᵢ of +tet cells incubated in Na-containing media decreased from 0.74±0.07 to 0.29±0.04 mM demonstrating SLC41A1-related Mg²⁺ extrusion (FIG. 2B). This Mg²⁺ efflux was clearly dependent on extracellular Na⁺ (FIG. 2). It decreased from 358±69 μM/20 min in 145-Na⁺ medium to 55±26 μM/20 min in 0-Na⁺ NMDG medium (FIG. 2C) reflecting a 82±5% reduction. These results clearly point to a Na-dependent mechanism of SLC41A1-related Mg²⁺ efflux.

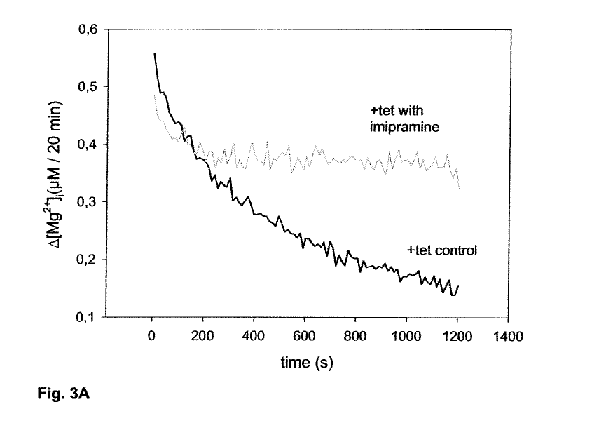

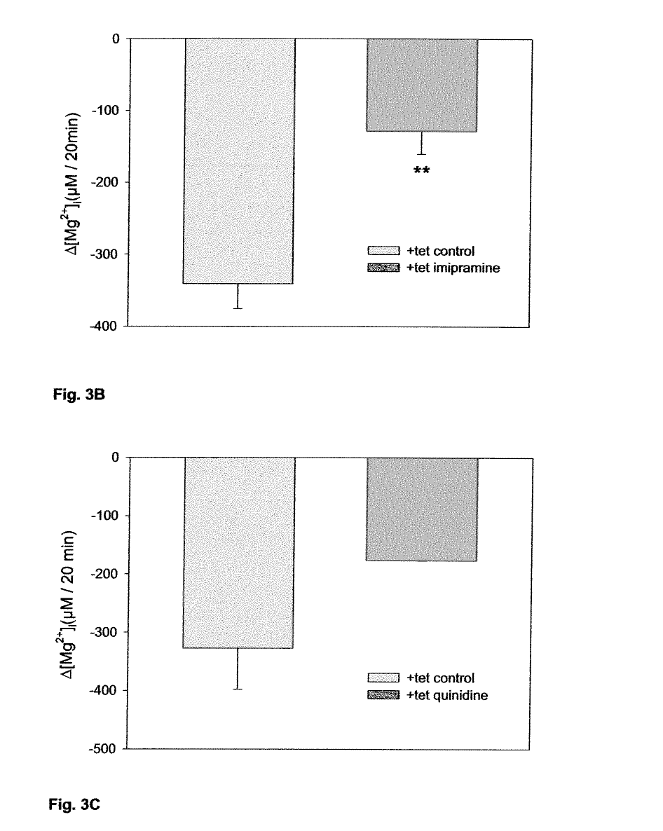

Therefore, we next evaluated whether application of known unspecific inhibitors of the Na⁺/Mg²⁺ exchanger resulted in changes of SLC41A1-mediated Mg²⁺ efflux. Mg²⁺-loaded (20 min, 10 mM) +tet HEK293-(SLC41A1) cells were incubated in a completely Mg²⁺-free medium with an [Na⁺]ₑ of 145 mM without or with 250 μM imipramine (FIG. 3A) or 100 μM quinidin (FIG. 3C), respectively, and the [Mg²⁺]ᵢ was measured for 20 min. To correct for molecular leakiness, control measurements were done with −tet cells. As shown in FIG. 3A, application of imipramine diminished the SLC41A1-related Mg²⁺ efflux. Data are summarized in FIG. 3B, which shows that inhibitor application reduced Mg²⁺ efflux from 341±35 to 129±32 μM/20 min corresponding to 64±9%. FIG. 3C shows a summary of the inhibitory effect of 100 μM quinidin on SLC41A1-mediated Mg²⁺ efflux. It decreased from 327±71 μM/20 min to 176±0.5 μM/20 min corresponding to a 38±7% reduction.

hSLC41A1-Mediated Mg²⁺ Efflux is Stimulated by dB-cAMP and Angiotensin II but Inhibited by Insulin.

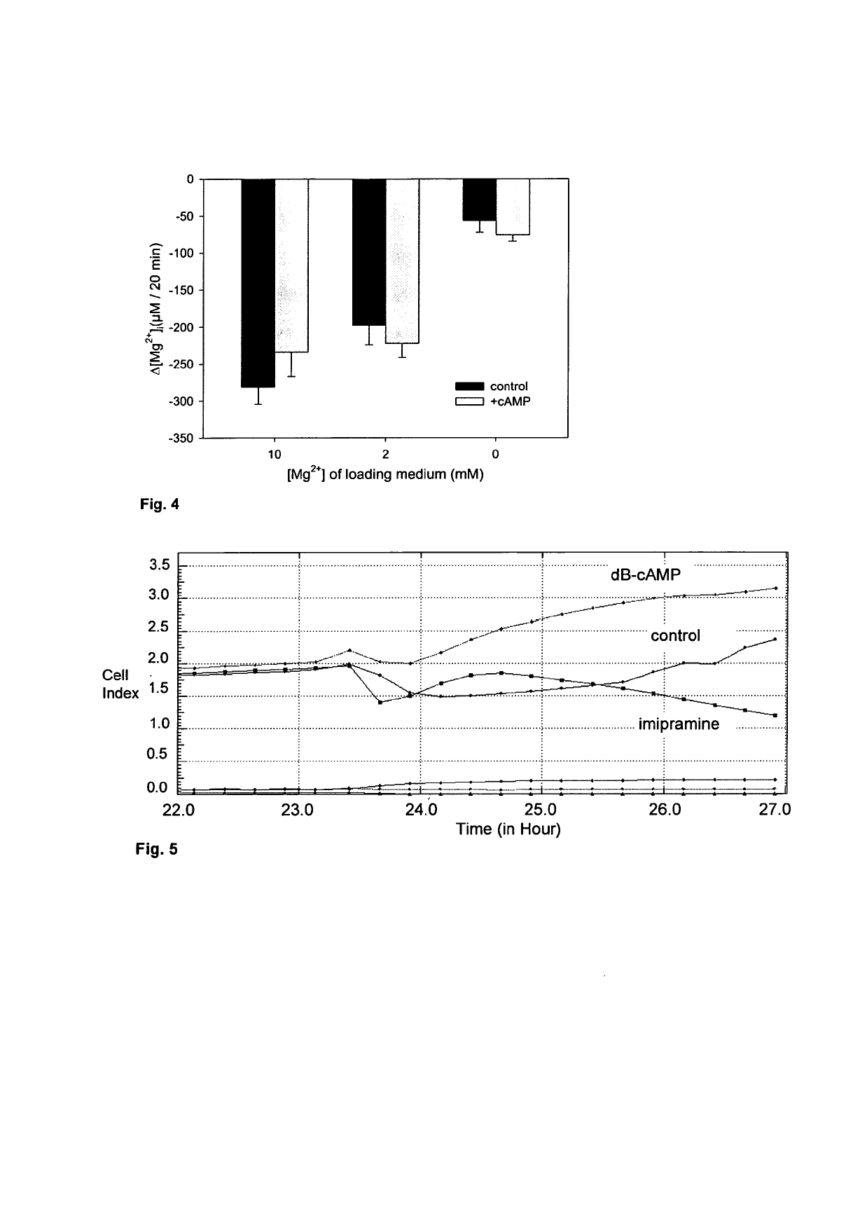

Depending on the loading conditions and thus, on the intracellular Mg²⁺ status, application of dB-cAMP decreases (loading in 10 mM-Mg²⁺ solution) SLC41A1-related Mg²⁺ efflux by 17% or increases (loading in 2 mM- and 0 mM-Mg²⁺ loading solution) it by 13% and 36%, respectively (FIG. 4). Interestingly, application of dB-cAMP to growing SLC41A1-overexpressing cells led to a stimulation of their proliferation (FIG. 5). In contrast, the Na⁺/Mg²⁺ exchanger inhibitor imipramin reduced the proliferation of such cells compared to controls (FIG. 5).

In addition, angiotensin II stimulated SLC41A1-related Mg²⁺ efflux by 19.5%.

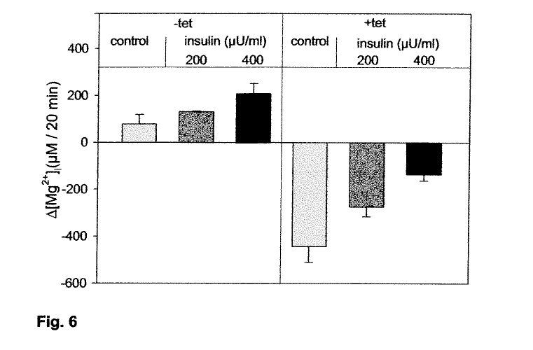

As shown in FIG. 6, insulin reduced the SLC41A1-mediated Mg²⁺ efflux by 38% (200 μU7ml) and 68% (400 μU/ml), respectively.

The Ca²⁺ Channel Inhibitor Verapamil has No Effect on hSLC41A1-Mediated Mg²⁺ Efflux.

In cardiac myocytes Verapamil has been show to effect Na⁺-dependent Mg²⁺ efflux by an unknown mechanism (Handy et al., 1996). Thus we tested the effect of this L-type Ca²⁺ channel inhibitor on Mg²⁺ efflux from +tet HEK293-(SLC41A1) cells. Although we used relatively high concentrations of 100 μM and 200 μM, Verapamil, respectively, we found no effect of the inhibitor on SLC41A1-mediated Mg²⁺ extrusion. The observed [Mg²⁺]ᵢ decrease amounted to 344±41 μM/20 min under control conditions and to 386±45 μM/20 min or 326±58 μM/20 min after application of 100 μM or 200 μM of verapamil.

### REFERENCES

1. Feil R. (2007). Conditional somatic mutagenesis in the mouse using site-specific recombinases. In Conditional Mutagenesis: An Approach to Disease Models, eds. R. Feil and D. Metzger, Springer-Verlag, Handb Exp Pharmacol 178, 3-28.
2. Goytain A, Quamme G A. Identification and characterization of a novel mammalian Mg²⁺ transporter with channel-like properties. BMC Genomics 2005a; 6: 48.
3. Goytain A, Quamme, G A. Functional characterization of ACDP2 (ancient conserved domain protein), a divalent metal transporter. Physiol Genomics 2005b; 22: 382-89.
4. Goytain, A.; Quamme G A. Functional characterization of the human solute carrier, SLC41A2. Biochem Biophys Res Commun 2005c; 330: 701-5
5. Goytain A, Quamme G A. Functional characterization of human SLC41A1, a Mg²⁺ transporter with similarity to the procaryotic MgtE Mg²⁺ transporters. Physiol Genomics 2005d; 21: 337-42.
6. Goytain A, Hines, R M, El-Husseini A, Quamme G A. NIPA1 (SPG6), the basis for autosomal dominant form of hereditary spastic paraplegia, encodes a functional Mg²⁺ transporter. J Biol Chem 2007; 282: 8060-68.
7. Günther T, Vormann J, Förster R (1984) Regulation of intracellular magnesium by Mg²⁺ efflux. Biochem Biophys Res Comm. 119: 124-131.
8. Handy R D, Gow I F, Ellis D, Flatman P W. Na-dependent regulation of intracellular free magnesium concentration in isolated rat ventricular myocytes. J Mol cell Cardiol 1996, 28: 1641-1651.
9. Kimura M (2007) Overview of magnesium nutrition. In: Nishizawa Y, Morii H, Durlach J (Eds.): New perspectives in magnesium research: nutrition and health. Springer, London, 2007, 69-93.
10. Kolisek M, Zsurka G, Samaj J, Weghuber J, Schweyen R J, Schweigel, M. Mrs2p is an essential component of the major electrophoretic Mg²⁺ influx system in mitochondria. EMBO J 2003; 22: 1235-44.
11. Kolisek M, Launay P, Beck A, Sponder G, Serafini M, Brenkus M, Froschauer E M, Martens H, Fleig A, Schweigel M. (2008) SLC41A1 is a novel mammalian Mg²⁺ carrier. J Biol Chem; 283: 16235-47.
12. Kolisek M, Schweyen R J, Schweigel M. (2007) Cellular Mg²⁺ transport and homeosasis: An overview. In: Nishizawa Y, Morii H, Durlach J (Eds.): New perspectives in magnesium research: nutrition and health. Springer, London, 2007, 21-33.
13. Kumeda Y, Inaba M. Review of magnesium and metabolic syndrome. In: Nishizawa Y, Morii H, Durlach J (Eds.): New perspectives in magnesium research: nutrition and health. Springer, London, 2007, 189-196.
14. Ödblom M P, Handy R D (1999) A novel DIDS-sensitive, anion-dependent Mg²⁺ efflux pathway in rat ventricular myocytes. Biochem. Biophys. Res. Comm. 264: 334-337.
15. Po S S; Wang D W, Yang I C, Johnson J P, Nie L, Bennett, P B. Modulation of HERG potassium channels by extracellular magnesium and quinidine. J Cardiovasc Pharmacot 1999, 33:181-185.
16. Quamme G A. (1989) Control of magnesium transport in the thick ascending limb. Am. J. Physiol. Renal Fluid Electrolyte Physiol. 25: F197-F210.
17. Resnick L M, Gupta R K, Laragh J H (1984) Intracellular free magnesium in erythrocytes of essential hypertension: relation to blood pressure and serum divalent cations. Proc Natl Acad Sci USA 81, 6511-6515.
18. Romanuik T L, Wang G, Holt R A; Jones S J M Marra M A, Sadar M D (2009) Identification of novel androgen-responsive genes by sequencing of LongSAGE libraries. BMC Genomics 10: 476-95.
19. Sahni J, Nelson B, Scharenberg A M. SLC41A2 encodes a plasma-membrane Mg²⁺ transporter. Biochem J 2007; 401: 505-13.
20. Schlingmann K P, Weber S, Peters M, Niemann-Nejsum L, Vitzhum H, Klingel K, Kratz M, Haddad E, Ristoff E, Dinour D, Syrrou M, Nielsen S, Sassen M, Waldegger S, Seyberth H W, Konrad M. Hypomagnesemia with secondary hypocalcemia is caused by mutations in TRPM6, a new member of the TRMP gene family. Nature Genetics 2002; 31: 166-70.
21. Schmitz C, Perraud A L, Johnson C O, Inabe K, Smith M K, Penner R, Kurosaki T, Fleig A, Scharenberg A M. Regulation of vertebrate cellular Mg²⁺-homeostasis by TRPM7. Cell 2003; 114: 191-200.
22. Schweigel M, Park H-S, Etschmann B, Martens H. Characterization of the Na⁺-dependent Mg²⁺ transport in sheep ruminal epithelial cells. Am J Physiol Gastrointest Liver Physiol 2006; 290: G56-G65.
23. Schweigel M, Park H-S, Etschmann B, Martens H. (2006) Characterization of the Na⁺-dependent Mg²⁺ transport in sheep ruminal epithelial cells. Am J Physiol Gastrointest Liver Physiol 290: G56-G65.
24. Smith, R. L., Thompson, L. J., and Maguire, M. E. (1995) J Bacteriol 177, 1233-8
25. Sponder G, Svidova S, Schweigel M, Vormann J, Kolisek M. (2010) Splice-variant 1 of the ancient domain protein 2 (ACDP2) complements the magnesium-deficient growth phenotype of Salmonella enterica sv. typhimurium strain MM281. Magnesium Research 23, 105-14.
26. Schweigel M, Vormann J, Martens H (2000) Mechanisms of Mg²⁺ transport in cultured rumen epithelial cells. Am J Physiol Gastrointest Liver Physiol 278: G400-G408.
27. Wabakken, T., Rian, E., Kveine, M., and Aasheim, H. C. (2003) Biochem Biophys Res Commun 306, 718-24
28. Watanabe M, Konishi M, Ohkido I, Matsufuji S. (2005) Enhanced sodium-dependent extrusion of magnesium in mutant cells established from a mouse renal tubular cell line. Am J Physiol Renal Physiol; 289, 742-48.
29. Zhou H, Clapham D E. (2009) Mammalian MagT1 and TUSC3 are required for cellular magnesium uptake and vertebrate embryonic development. Proc Natl Acad Sci USA.: 106, 15750-5.
30. Johnsson and Varshaysky, Proc. Natl. Acad. Sci., 1994, 91 (22), 10340-10344, in U.S. Pat. No. 5,503,977 A or U.S. Pat. No. 5,585,245 A.
31. Shibahara Kei-ichi et al. in published US patent application 20100003686

1. Mutation library for the identification or generation of SLC41A1 variants, wherein the protein SLC41A1 having the amino acid sequence of SEQ ID NO 1 is the cellular Na⁺/Mg²⁺ exchanger, comprising
i) proteins with mutations of SEQ ID NO 1, wherein one of the amino acids of SEQ ID NO 1 is substituted at positions 21 (S>N), 25 (S>Q), 73 (S>N), 76 (S>N), 80 (S>N), 89 (S>N), 97 (S>N), 158 (S>N), 309 (S>N), 394 (S>N), 408 (S>Q), 81 (T>Q), 168 (T>Q), 509 (T>Q) or 278 (Y>N) (15 mutated proteins) and at least one protein with mutation of SEQ ID NO 1, wherein a plurality of these substitutions is present
or
ii) proteins with mutations of the transmembrane domains and N-, C-termini mutants of SEQ ID NO 1, wherein the amino acids 2-30, 31-60, 61-90, 2-60, 2-90 or 504-513 of SEQ ID NO 1 are deleted (6 mutated proteins)
and
proteins with mutations of SEQ ID NO 1 in membrane spanning domains, wherein the amino acids 96-205, 225-279, 286-337, 348-427 or 439-503 are deleted (5 mutated proteins)
and proteins with mutations in membrane spanning domains of SEQ ID NO 1, wherein the amino acids 99-150, 180-241, 256-306, 317-368, 408-459 or 437-503 are deleted (6 mutated proteins)
or
iii) proteins with leucine zipper (LZ) mutants of SEQ ID NO 1, wherein the amino acids 482-503 are deleted and wherein amino acids in LZ domain in positions 482 (L>V), 489 (L>V), 494 (L>V), 496 (L>V), 501 (L>V) or 503 (L>V) are substituted (7 mutated proteins)
or
iv) proteins with external loop (EL) mutants of SEQ ID NO 1, wherein the amino acids 128-179, 247-254, 310-315, 369-405, or 468-478 are deleted (5 mutated proteins).

2. Transgenic inducible conditional knock out non-human mammal characterized in that the genome comprises a construct, which allows inducible conditional knock out of the SCL41A1 gene, whose corresponding protein SLC41A1 is the cellular Na⁺/Mg²⁺ exchanger, wherein the construct comprises the SCL41A1 gene of SEQ ID NO 4 flanked by two loxP DNA sequence elements and in that the non-human mammal expresses Cre recombinase or ligand-dependent Cre recombinase.

3. Cells from the transgenic inducible conditional knock out non-human mammal according to claim 2, which additionally has in its genome a mammalian vector with a mutated human SLC41A1 sequence of SEQ ID NO 1 selected from the mutation library according to claim 1.

4. Transgenic cell line having inducible conditionally knocked out SCL41A1 gene, whose corresponding protein SLC41A1 is the cellular Na⁺/Mg²⁺ exchanger, which has in its genome a mammalian vector with a mutated human SLC41A1 sequence of SEQ ID NO 1 selected from the mutation library according to claim 1.
